(19) United States
(12) Patent Application Publication
(10) Pub. No.: US 2016/0166676 A1
(43) Pub. Date: Jun. 16, 2016

(54) Use of Immune Suppressive Peptides as Adjuvants

(71) Applicant: SKAU APS, Aarhus N (DK)

(72) Inventor: Shervin BAHRAMI, Aarhus C (DK)

(21) Appl. No.: 14/783,280

(22) PCT Filed: Apr. 10, 2014
(86) PCT No.: PCT/DK2014/050090

§ 371 Date: Oct. 8, 2015

(30) Foreign Application Priority Data
Apr. 10, 2013 (DK) PA 2013 70200

(51) Int. Cl. A61K 39/145; A61K 39/39; C12N 7/00

(52) U.S. Cl. CPC A61K 39/145; C12N 7/00; A61K 39/39; A61K 2039/55516

(57) ABSTRACT

The present invention relates to an adjuvant comprising at least one immunosuppressive domain for use in a vaccine.

The present invention relates to adjuvants for use in vaccines. In particular, the present invention relates to an adjuvant comprising at least one immunosuppressive domain for use in a vaccine.

### TECHNICAL BACKGROUND

Typically, in viruses one or mores transmembrane glycoproteins, fusion proteins, undergoe a conformational transition triggered by receptor recognition or low pH, leading to the insertion of a fusion peptide into the plasma membrane or the membrane of an endocytic vesicle. For some viruses, for example members of the paramyxovirus family, separate envelope proteins mediate attachment and fusion.

Membrane fusion can occur either at the plasma membrane or at an intracellular location following internalization of virus by receptor-mediated endocytosis. Fusion is mediated by viral transmembrane proteins known as fusion proteins. Upon appropriate triggering, the fusion protein interacts with the target membrane through a hydrophobic fusion peptide and undergoes a conformational change that drives the membrane fusion reaction. There are a variety of fusion triggers, including various combinations of receptor binding, receptor/coreceptor binding, and exposure to the mildly acidic pH within the endocytic pathway. Fusion proteins from different viruses have different names in spite of the common functionality.

Based on important structural features, many virus membrane fusion proteins are currently annotated to either the “class I” membrane fusion proteins exemplified by the influenza hemagglutinin (HA) or HIV-1 gp41, or the “class II” proteins of the alphaviruses and flaviviruses. The alphaviruses and flaviviruses are members of the Togaviridae and Flaviviridae families, respectively. These small enveloped positive-sense RNAviruses are composed of a capsid protein that assembles with the RNA into the nucleocapsid, and a lipid bilayer containing the viral transmembrane (TM) proteins.

Class I fusion proteins are synthesized as single chain precursors, which then assemble into trimers. The polypeptides are then cleaved by host proteases, which is an essential step in rendering the proteins fusion competent. This proteolytic event occurs late in the biosynthetic process because the fusion proteins, once cleaved are metastable and readily activated. Once activated, the protein refolds into a highly stable conformation. The timing of this latter event is of crucial importance in the fusion process. Maintenance of the intact precursor polypeptide during folding and assembly of the oligomeric structure is essential if the free energy that is released during the refolding event is to be available to overcome the inherent barriers to membrane fusion. The new amino-terminal region that is created by the cleavage event contains a hydrophobic sequence, which is known as the fusion peptide. The authentic carboxy-terminal region of the precursor polypeptide contains the transmembrane anchor. In the carboxy-terminal polypeptide, there are sequences known as the heptad repeat that are predicted to have an alpha helical structure and to form a coiled coil structure. These sequences participate in the formation of highly stable structure that characterizes the post-fusion conformation of the fusion protein.

The class II fusion proteins are elongated finger-like molecules with three globular domains composed almost entirely of β-sheets. Domain I is a β-barrel that contains the N-terminus and two long insertions that connect adjacent β-strands and together form the elongated domain II. The first of these insertions contains the highly conserved fusion peptide loop at its tip, connecting the c and d β-strands of domain II (termed the cd loop) and containing 4 conserved disulfide bonds including several that are located at the base of the fusion loop. The second insertion contains the ij loop at its tip, adjacent to the fusion loop, and one conserved disulfide bond at its base. A hinge region is located between domains I and II. A short linker region connects domain I to domain III, a β-barrel with an immunoglobulin-like fold stabilized by three conserved disulfide bonds. In the full-length molecule, domain III is followed by a stem region that connects the protein to the virus TM anchor. Fitting of the structure of alphavirus E1 to cryo-electron microscopy reconstructions of the virus particle reveals that E1 is located almost parallel to the virus membrane, and that E1-E1-interactions form the an icosahedral lattice.

### Fusion Peptides

Fusion peptides are moderately hydrophobic segments of viral and non-viral membrane fusion proteins that enable these proteins to disrupt and connect two closely apposed biological membranes. This process, which results in membrane fusion occurs in a well-controlled manner with a surprisingly small amount of leakage of the contents of the encapsulated volumes to the outside world. The sequences of fusion peptides are highly conserved within different groups of fusion proteins, for example within different virus families, but not between them. Most fusion peptides are located at the extreme N-termini of the transmembrane subunits of the fusion proteins. However, in a few cases such as the sperm protein fertilin-α, vesicular stomatitis virus G, baculovirus gp64, and Rous sarcoma virus gp37, internal fusion peptides have been found. Deletion of the fusion peptide and, in many cases, even relatively conservative single amino acid changes in the fusion peptide completely abolish the ability of fusion proteins to fuse membranes, while other structural and functional properties of these proteins may remain intact. Conversely, single amino acid changes in many other regions of these proteins are less deleterious to their function. Such mutagenesis experiments clearly point to a central role of the fusion peptides in membrane fusion. It has further been shown in a number of cases that even isolated fusion peptides alone can support membrane fusion in model systems. (Tamm and Han, Bioscience Reports, Vol. 20, No. 6, 2000).

### Immune Suppressive Domains—Immunosuppressive Properties of Enveloped Viruses

Fusion proteins of a subset of enveloped Type I viruses (retrovirus, lentivirus and filoviruses) have previously been shown to feature an immune suppressive activity. Inactivated retroviruses are able to inhibit proliferation of immune cells upon stimulation. Expression of these proteins is enough to enable allogenic cells to grow to a tumor in immune competent mice. In one study, introduction of ENV expressing construct into MCA205 murine tumor cells, which do not proliferate upon s.c. injection into an allogeneic host, or into CL8.1 murine tumor cells (which overexpress class I antigens and are rejected in a syngeneic host) resulted in tumor growth in both cases. Such immunosuppressive domains have been found in a variety of different viruses with type 1 fusion mechanism such as gamma-retroviruses like Mason pfeizer monkey virus (MPMV) and murine leukemia virus (MLV), lentiviruses such as HIV and in filoviruses such as Ebola and Marburg viruses.

This immune suppressive activity was in all cases located to a very well-defined structure within the class I fusion proteins, more precisely at the bend in the heptad repeat just N-terminale of the transmembrane structure in the fusion protein. The immunosuppressive effects range from significant inhibition of lymphocyte proliferation, cytokine skewing (up regulating IL-10; down regulating TNF-α, IL-12, IFN-γ) and inhibition of monocytic burst to cytotoxic T cell killing. Importantly, peptides spanning ISD in these assays must either be linked as dimers or coupled to a carrier (i.e. >monomeric) to be active. Such peptides derived from immune-suppressive domains are able to reduce or abolish immune responses such as cytokine secretion or proliferation of T-cells upon stimulation. The protection mediated by the immunosuppressive properties of the fusion protein from the immune system of the host is not limited to the fusion protein but covers all the viral envelope proteins displayed at viral or cellular membranes in particular also the protein mediating attachment of the virus to the cell.

### Co-Location of the Immunosuppression Domain and the Fusion Domain

The immunosuppressive domains of viruses like but not limited to retro-, lenti-, Orthomyxo-, flavi- and filoviruses overlap structurally important parts of the fusion subunits of the surface glycoproteins. In several cases the primary structure (sequence) of the ISD can vary greatly from virus to virus, but the secondary structure, which is very well preserved among different virus families, is that of an alpha helix that bends in different ways during the fusion process This structure plays a crucial role during events that result in fusion of viral and cellular membranes. It is evident that the immunosuppressive domains of these (retroviral, lentiviral and filoviral) class I fusion proteins overlap with a very important protein structure needed for the fusion mechanistic function.

The energy needed for mediating the fusion of viral and cellular membranes is stored in the fusion proteins, which are thus found in a meta-stable conformation on the viral surface. Once the energy is released to drive the fusion event, the protein will find its most energetically stable conformation. In this regard fusion proteins can be compared with loaded springs that are ready to be sprung. This high energy conformation makes the viral fusion proteins very susceptible to modifications; Small changes in the primary structure of the protein often result in the protein to be folded in its stable post fusion conformation. The two conformations present very different tertiary structures of the same protein.

It has been shown in the case of simple retroviruses that small structural changes in the envelope protein are sufficient to remove the immune suppressive effect without changing structure and hence the antigenic profile.

The mutated non-immune suppressive envelope proteins are much better antigens for vaccination. The proteins can induce a 30-fold enhancement of anti-env antibody titers when used for vaccination and are much better at launching an effective CTL response. Furthermore, viruses that contain the non-immunosuppressive form of the friend murine leukemia virus envelope protein, although fully infectious in irradiated immunocompromised mice cannot establish an infection in immunocompetent animals. Interestingly in the latter group the non-immunosuppressive viruses induce both a higher cellular and humeral immune response, which fully protect the animals from subsequent challenge by wild type viruses.

Immunosuppressive domains in the fusion proteins (viral envelope proteins) from Retroviruses, lentiviruses and Filoviruses have been known since 1985 for retrovirus, since 1988 for lentivirus and since 1992 for filoviruses. These viruses, as mentioned above, all belong to enveloped RNA viruses with a type I fusion mechanism. The immunosuppressive domains of lentivirus, retroviruses and filoviruses show large structural similarity. Furthermore the immunosuppressive domain of these viruses are all located at the same position in the structure of the fusion protein, more precisely in the linker between the two heptad repeat structures just N-terminal of the transmembrane domain in the fusion protein. These heptad repeat regions constitute two alpha helices that play a critical role in the active mechanism of membrane fusion by these proteins. The immune suppressive domains can be located in relation to two well conserved cystein residues that are found in these structures. These cystein residues are between 4 and 6 amino acid residues from one another and in many cases are believed to form disulfide bridges that stabilize the fusion proteins. The immune suppressive domains in all three cases include at least some of the first 22 amino acids that are located N-terminal to the first cysteine residue. Recently the immunosuppressive domains in the fusion protein of these viruses have been successfully altered in such a way that the fusogenic properties of the fusion protein have been preserved. Such mutated fusion proteins with decreased immunosuppressive properties have been shown to be superior antigens for vaccination purposes.

Other immunosuppressive domains are found in type II fusion proteins. Immunosuppressive domains have been identified at different positions in different groups of viruses. For example an immune suppressive domain might co-localize with the fusion peptide exemplified by the identification of an common immunosuppressive domain in the fusion peptide of Flavirius (Dengue virus, west Nile virus etc), or with the hydrophobic alpha helix N-terminal of the transmembrane domain in the fusion protein exemplified by the finding of an immunosuppressive domain in said helixes of all flaviridae e.g. Hepatitis C virus, Dengue, west nile etc.

The immune suppressive domains can also be located in the fusion peptide of the fusion protein among enveloped RNA viruses with type I fusion mechanism. For example HIV or influenza A and B types have an immune suppressive domain that co-localized with their fusion peptide.

Immunosuppressive domains are identified among enveloped RNA viruses with type II fusion mechanism at different positions in different groups of viruses:
i. Co-localizing with the fusion peptide exemplified by the identification of an common immunosuppressive domain in the fusion peptide of Flavirius (Dengue virus, west Nile virus etc), and
ii. In the hydrophobic alpha helix N-terminal of the transmembrane domain in the fusion protein exemplified by the finding of an immunosuppressive domain in said helixes of all flaviridae e.g. Hepatitis C virus, Dengue, west nile etc.

2: Immunosuppressive domains have been identified in the fusion protein among enveloped RNA viruses with type I fusion mechanism. This position co-localizes with the fusion peptide of said fusion protein as demonstrated by the identification of a common immunosuppressive domain in the fusion peptide of all Influenza A and B types as well as HIV.

### Membrane Fusion and STING Pathway

Virus-cell fusion specifically stimulate a type I interferon response with expression of interferon-stimulated genes, in vivo recruitment of leukocytes and potentiation of signaling via Toll-like receptor 7 (TLR7) and TLR9. The fusion-dependent response is dependent on the stimulator of interferon genes STING.

The molecule referred to as STING (stimulator of interferon genes) also known as known as MITA/MPYS/ERIS is also essential for cytosolic DNA-mediated type I IFNs induction. STING contains multi-putative transmembrane regions in the amino terminal region, and is found to associate with membranes.

The existence of immune suppressive domains in the viral fusion proteins is expected to insert the immune suppressive activity partly through interference with this pathway either through direct or indirect interaction with STING, Hence an antagonist of this putative interaction will enhance the immune responses to proteins containing such immune suppressive domains and can be used as adjuvants

### Functional Homolog

The term “functional homologue” or “functional equivalent” refers to homologues of the molecules according to the present invention and is meant to comprise any molecule which is capable of mimicking the function of molecules as described herein. Thus, the terms refer to functional similarity or, interchangeably, functional identity, between two or more molecular entities. The term “functional homology” is further used herein to describe that one molecular entity are able to mimic the function of one or more molecular entities.

Functional homologues according to the present invention may comprise any molecule that can function as an antagonist of the immune suppressive activity exerted by an immune suppressive domains. Such a molecule when added to the composition containing said immune suppressive domains reduces the immune suppressive activity exerted by the latter in either an in vitro test system (e.g. CTLL-2 or PBMC proliferation assays) or in vivo seen as an enhanced T- and/or B-cell responses.

Functional homologues according to the present invention may comprise polypeptides with an amino acid sequence, which are sharing at least some homology with the predetermined polypeptide sequences as outlined herein. For example such polypeptides are at least about 40 percent, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with the predetermined polypeptide sequences as outlined herein above. The homology between amino acid sequences may be calculated using well known algorithms such as for example any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Functional homologues may comprise an amino acid sequence that comprises at least one substitution of one amino acid for any other amino acid. For example such a substitution may be a conservative amino acid substitution or it may be a non-conservative substitution. A conservative amino acid substitution is a substitution of one amino acid within a predetermined group of amino acids for another amino acid within the same group, wherein the amino acids within predetermined groups exhibit similar or substantially similar characteristics. Within the meaning of the term “conservative amino acid substitution” as applied herein, one amino acid may be substituted for another within groups of amino acids characterized by having
i) hydrophilic (polar) side chains (Asp, Glu, Lys, Arg, His, Asn, Gln, Ser, Thr, Tyr, and Cys,)
ii) hydrophobic (non-polar) side chains (Gly, Ala, Val, Leu, Ile, Phe, Trp, Pro, and Met)
iii) aliphatic side chains (Gly, Ala Val, Leu, Ile)
iv) cyclic side chains (Phe, Tyr, Trp, His, Pro)
v) aromatic side chains (Phe, Tyr, Trp)
vi) acidic side chains (Asp, Glu)
vii) basic side chains (Lys, Arg, His)
viii) amide side chains (Asn, Gln)
ix) hydroxy side chains (Ser, Thr)
x) sulphor-containing side chains (Cys, Met), and
xi) amino acids being monoamino-dicarboxylic acids or monoamino-monocarboxylic-monoamidocarboxylic acids (Asp, Glu, Asn, Gln).

Non-conservative substitutions are any other substitutions. A non-conservative substitution leading to the formation of a functional homologue would for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Functional homologues according to the present invention may comprise more than one such substitution, such as e.g. two amino acid substitutions, for example three or four amino acid substitutions, such as five or six amino acid substitutions, for example seven or eight amino acid substitutions, such as from 10 to 15 amino acid substitutions, for example from 15 to 25 amino acid substitution, such as from 25 to 30 amino acid substitutions, for example from 30 to 40 amino acid substitution, such as from 40 to 50 amino acid substitutions, for example from 50 to 75 amino acid substitution, such as from 75 to 100 amino acid substitutions, for example more than 100 amino acid substitutions. The addition or deletion of an amino acid may be an addition or deletion of from 2 to 5 amino acids, such as from 5 to 10 amino acids, for example from 10 to 20 amino acids, such as from 20 to 50 amino acids. However, additions or deletions of more than 50 amino acids, such as additions from 50 to 200 amino acids, are also comprised within the present invention. The polypeptides according to the present invention, including any variants and functional homologues thereof, may in one embodiment comprise more than 5 amino acid residues, such as more than 10 amino acid residues, for example more than 20 amino acid residues, such as more than 25 amino acid residues, for example more than 50 amino acid residues, such as more than 75 amino acid residues, for example more than 100 amino acid residues, such as more than 150 amino acid residues, for example more than 200 amino acid residues.

### Genetic Code

The genetic code is the set of rules by which information encoded within genetic material (DNA or mRNA sequences) is translated into proteins (amino acid sequences) by living cells. Biological decoding is accomplished by the ribosome, which links amino acids in an order specified by mRNA, using transfer RNA (tRNA) molecules to carry amino acids and to read the mRNA three nucleotides at a time. The genetic code is highly similar among all organisms, and can be expressed in a simple table with 64 entries.

The code defines how sequences of these nucleotide triplets, called codons, specify which amino acid will be added next during protein synthesis. With some exceptions a three-nucleotide codon in a nucleic acid sequence specifies a single amino acid. Because the vast majority of genes are encoded with exactly the same code (see the RNA codon table), this particular code is often referred to as the canonical or standard genetic code, or simply the genetic code, though in fact some variant codes have evolved. For example, protein synthesis in human mitochondria relies on a genetic code that differs from the standard genetic code.

Not all genetic information is stored using the genetic code. All organisms' DNA contains regulatory sequences, intergenic segments, chromosomal structural areas, and other non-coding DNA that can contribute greatly to phenotype. Those elements operate under sets of rules that are distinct from the codon-to-amino acid paradigm underlying the genetic code.

Genetically encoded amino acids are as described below. Any other amino acid except for the 20 described below is considered a non-genetically encoded amio acid.

| Amino | | |
|---|---|---|
| acid | Codons | Compressed |
| Ala/A | GCU, GCC, GCA, | GCN |
| | GCG | |
| Arg/R | CGU, CGC, CGA, | CGN, MGR |
| | CGG, AGA, AGG | |
| Asn/N | AAU, AAC | AAY |
| Asp/D | GAU, GAC | GAY |
| Cys/C | UGU, UGC | UGY |
| Gln/Q | CAA, CAG | CAR |
| Glu/E | GAA, GAG | GAR |
| Gly/G | GGU, GGC, GGA, | GGN |
| | GGG | |
| His/H | CAU, CAC | CAY |
| Ile/I | AUU, AUC, AUA | AUH |
| Leu/L | UUA, UUG, CUU, | YUR, CUN |
| | CUC, CUA, CUG | |
| Lys/K | AAA, AAG | AAR |
| Met/M | AUG | |
| Phe/F | UUU, UUC | UUY |
| Pro/P | CCU, CCC, CCA, | CCN |
| | CCG | |
| Ser/S | UCU,UCC,UCA, | UCN, AGY |
| | UCG, AGU, AGC | |
| Thr/T | ACU, ACC, ACA, | ACN |
| | ACG | |
| Trp/W | UGG | |
| Tyr/Y | UAU, UAC | UAY |
| Val/V | GUU, GUC, GUA, | GUN |
| | GUG | |

### D- and L-Amino Acids

Of the standard α-amino acids, all but glycine can exist in either of two enantiomers, called L or D amino acids, which are mirror images of each other. While L-amino acids represent all of the amino acids found in proteins during translation in the ribosome, D-amino acids are found in some proteins produced by enzyme posttranslational modifications after translation and translocation to the endoplasmic reticulum, as in exotic sea-dwelling organisms such as cone snails. They are also abundant components of the peptidoglycan cell walls of bacteria, and D-serine may act as a neurotransmitter in the brain. The L and D convention for amino acid configuration refers not to the optical activity of the amino acid itself, but rather to the optical activity of the isomer of glyceraldehyde from which that amino acid can, in theory, be synthesized (D-glyceraldehyde is dextrorotary; L-glyceraldehyde is levorotatory).

### Lipids

Lipids constitute a group of naturally occurring molecules that include fats, waxes, sterols, fat-soluble vitamins (such as vitamins A, D, E, and K), monoglycerides, diglycerides, triglycerides, phospholipids, and others. Lipids may belong to the following categories.

### Fatty Acids

Fatty acids, or fatty acid residues when they form part of a lipid, are a diverse group of molecules synthesized by chain-elongation of an acetyl-CoA primer with malonyl-CoA or methylmalonyl-CoA groups in a process called fatty acid synthesis. They are made of a hydrocarbon chain that terminates with a carboxylic acid group; this arrangement confers the molecule with a polar, hydrophilic end, and a nonpolar, hydrophobic end that is insoluble in water. The fatty acid structure is one of the most fundamental categories of biological lipids, and is commonly used as a building-block of more structurally complex lipids. The carbon chain, typically between four and 24 carbons long, may be saturated or unsaturated, and may be attached to functional groups containing oxygen, halogens, nitrogen, and sulfur. Where a double bond exists, there is the possibility of either a cis or trans geometric isomerism, which significantly affects the molecule's configuration. Cis-double bonds cause the fatty acid chain to bend, an effect that is compounded with more double bonds in the chain. This in turn plays an important role in the structure and function of cell membranes. Most naturally occurring fatty acids are of the cis configuration, although the trans form does exist in some natural and partially hydrogenated fats and oils.

Examples of biologically important fatty acids are the eicosanoids, derived primarily from arachidonic acid and eicosapentaenoic acid, that include prostaglandins, leukotrienes, and thromboxanes. Docosahexaenoic acid is also important in biological systems, particularly with respect to sight. Other major lipid classes in the fatty acid category are the fatty esters and fatty amides. Fatty esters include important biochemical intermediates such as wax esters, fatty acid thioester coenzyme A derivatives, fatty acid thioester ACP derivatives and fatty acid carnitines. The fatty amides include N-acyl ethanolamines, such as the cannabinoid neurotransmitter anandamide.

### Glycerolipids

Glycerolipids are composed mainly of mono-, di-, and tri-substituted glycerols, the most well-known being the fatty acid triesters of glycerol, called triglycerides. The word “triacylglycerol” is sometimes used synonymously with “triglyceride”, though the latter lipid contains no hydroxyl group. In these compounds, the three hydroxyl groups of glycerol are each esterified, typically by different fatty acids. Because they function as an energy store, these lipids comprise the bulk of storage fat in animal tissues. The hydrolysis of the ester bonds of triglycerides and the release of glycerol and fatty acids from adipose tissue are the initial steps in metabolising fat.

Additional subclasses of glycerolipids are represented by glycosylglycerols, which are characterized by the presence of one or more sugar residues attached to glycerol via a glycosidic linkage. Examples of structures in this category are the digalactosyldiacylglycerols found in plant membranes and seminolipid from mammalian sperm cells.

### Glycerophospholipids

Glycerophospholipids, usually referred to as phospholipids, are ubiquitous in nature and are key components of the lipid bilayer of cells, as well as being involved in metabolism and cell signaling. Neural tissue (including the brain) contains relatively high amounts of glycerophospholipids, and alterations in their composition has been implicated in various neurological disorders. Glycerophospholipids may be subdivided into distinct classes, based on the nature of the polar headgroup at the sn-3 position of the glycerol backbone in eukaryotes and eubacteria, or the sn-1 position in the case of archaebacteria.

Examples of glycerophospholipids found in biological membranes are phosphatidylcholine (also known as PC, GPCho or lecithin), phosphatidylethanolamine (PE or GPEtn) and phosphatidylserine (PS or GPSer). In addition to serving as a primary component of cellular membranes and binding sites for intra- and intercellular proteins, some glycerophospholipids in eukaryotic cells, such as phosphatidylinositols and phosphatidic acids are either precursors of or, themselves, membrane-derived second messengers. Typically, one or both of these hydroxyl groups are acylated with long-chain fatty acids, but there are also alkyl-linked and 1Z-alkenyl-linked (plasmalogen) glycerophospholipids, as well as dialkylether variants in archaebacteria.

### Sphingolipids

Sphingolipids are a complicated family of compounds that share a common structural feature, a sphingoid base backbone that is synthesized de novo from the amino acid serine and a long-chain fatty acyl CoA, then converted into ceramides, phosphosphingolipids, glycosphingolipids and other compounds. The major sphingoid base of mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms.

The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

### Sterol Lipids

Sterol lipids, such as cholesterol and its derivatives, are an important component of membrane lipids, along with the glycerophospholipids and sphingomyelins. The steroids, all derived from the same fused four-ring core structure, have different biological roles as hormones and signaling molecules. The eighteen-carbon (C18) steroids include the estrogen family whereas the C19 steroids comprise the androgens such as testosterone and androsterone. The C21 subclass includes the progestogens as well as the glucocorticoids and mineralocorticoids. The secosteroids, comprising various forms of vitamin D, are characterized by cleavage of the B ring of the core structure. Other examples of sterols are the bile acids and their conjugates, which in mammals are oxidized derivatives of cholesterol and are synthesized in the liver. The plant equivalents are the phytosterols, such as β-sitosterol, stigmasterol, and brassicasterol; the latter compound is also used as a biomarker for algal growth. The predominant sterol in fungal cell membranes is ergosterol.

### Prenol Lipids

Prenol lipids are synthesized from the five-carbon-unit precursors isopentenyl diphosphate and dimethylallyl diphosphate that are produced mainly via the mevalonic acid (MVA) pathway. The simple isoprenoids (linear alcohols, diphosphates, etc.) are formed by the successive addition of C5 units, and are classified according to number of these terpene units. Structures containing greater than 40 carbons are known as polyterpenes. Carotenoids are important simple isoprenoids that function as antioxidants and as precursors of vitamin A. Another biologically important class of molecules is exemplified by the quinones and hydroquinones, which contain an isoprenoid tail attached to a quinonoid core of non-isoprenoid origin. Vitamin E and vitamin K, as well as the ubiquinones, are examples of this class. Prokaryotes synthesize polyprenols (called bactoprenols) in which the terminal isoprenoid unit attached to oxygen remains unsaturated, whereas in animal polyprenols (dolichols) the terminal isoprenoid is reduced.

### Saccharolipids

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

### Polyketides

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, and/or other processes. Many commonly used anti-microbial, anti-parasitic, and anti-cancer agents are polyketides or polyketide derivatives, such as erythromycins, tetracyclines, avermectins, and antitumor epothilones.

### Biological Functions in Membranes

Eukaryotic cells are compartmentalized into membrane-bound organelles that carry out different biological functions. The glycerophospholipids are the main structural component of biological membranes, such as the cellular plasma membrane and the intracellular membranes of organelles; in animal cells the plasma membrane physically separates the intracellular components from the extracellular environment. The glycerophospholipids are amphipathic molecules (containing both hydrophobic and hydrophilic regions) that contain a glycerol core linked to two fatty acid-derived “tails” by ester linkages and to one “head” group by a phosphate ester linkage. While glycerophospholipids are the major component of biological membranes, other non-glyceride lipid components such as sphingomyelin and sterols (mainly cholesterol in animal cell membranes) are also found in biological membranes. In plants and algae, the galactosyldiacylglycerols, and sulfoquinovosyldiacylglycerol, which lack a phosphate group, are important components of membranes of chloroplasts and related organelles and are the most abundant lipids in photosynthetic tissues, including those of higher plants, algae and certain bacteria.

Bilayers have been found to exhibit high levels of birefringence, which can be used to probe the degree of order (or disruption) within the bilayer using techniques such as dual polarization interferometry and Circular dichroism.

A biological membrane is a form of lipid bilayer. The formation of lipid bilayers is an energetically preferred process when the glycerophospholipids described above are in an aqueous environment. This is known as the hydrophobic effect. In an aqueous system, the polar heads of lipids align towards the polar, aqueous environment, while the hydrophobic tails minimize their contact with water and tend to cluster together, forming a vesicle; depending on the concentration of the lipid, this biophysical interaction may result in the formation of micelles, liposomes, or lipid bilayers. Other aggregations are also observed and form part of the polymorphism of amphiphile (lipid) behavior. Phase behavior is an area of study within biophysics and is the subject of current academic research. Micelles and bilayers form in the polar medium by a process known as the hydrophobic effect. When dissolving a lipophilic or amphiphilic substance in a polar environment, the polar molecules (i.e., water in an aqueous solution) become more ordered around the dissolved lipophilic substance, since the polar molecules cannot form hydrogen bonds to the lipophilic areas of the amphiphile. So in an aqueous environment, the water molecules form an ordered “clathrate” cage around the dissolved lipophilic molecule.

### Adjuvant

An adjuvant (from Latin, adiuvare: to aid) is a pharmacological or immunological agent that modifies the effect of other agents, such as a drug or vaccine. They are often included in vaccines to enhance the recipient's immune response to a supplied antigen, while keeping the injected foreign material to a minimum.

### Immunologic Adjuvants

In immunology, an adjuvant is an agent that may stimulate the immune system and increase the response to a vaccine, without having any specific antigenic effect in itself. An immunologic adjuvant is defined as any substance that acts to accelerate, prolong, or enhance antigen-specific immune responses when used in combination with specific vaccine antigens.”. There are many known adjuvants in widespread use, including oils, aluminium salts, and virosomes.

Immunologic adjuvants are added to vaccines to stimulate the immune system's response to the target antigen, but do not in themselves confer immunity. Adjuvants can act in various ways in presenting an antigen to the immune system. Adjuvants can act as a depot for the antigen, presenting the antigen over a long period of time, thus maximizing the immune response before the body clears the antigen. Examples of depot type adjuvants are oil emulsions. Adjuvants can also act as an irritant which causes the body to recruit and amplify its immune response. A tetanus, diphtheria, and pertussis vaccine, for example, contains minute quantities of toxins produced by each of the target bacteria, but also contains some aluminium hydroxide. Such aluminium salts are common adjuvants in vaccines sold in the United States and have been used in vaccines for over 70 years. The body's immune system develops an antitoxin to the bacteria's toxins, not to the aluminium, but would not respond enough without the help of the aluminium adjuvant.

### SUMMARY OF THE INVENTION

The inventors speculate that the immune suppressive domains of viral surface proteins act through interaction with cellular components to reduce or abolish the induction of immune responses. Hence an antagonist of the cellular interaction partners of immune suppressive domains will abolish the suppression activity and induce higher immune responses accordingly. Such a molecule may act as an adjuvant which will enhance the efficacy of vaccines.

In one aspect the monomeric forms of the immune suppressive domain derived peptides will function as adjuvants. It appears that the immune suppressive domains show immune suppressive activity only as dimer or mulitmers in concordance with the fact that viral fusion proteins (form which the ISDs are derived) are usually trimers, sometimes dimers but are never found in monomeric form. The monomeric peptides corresponding to the immune suppressive domains show no immune suppressive activity in vitro, but they can interact with the relevant cellular components blocking the interaction sites for dimer or mulitimeric functional peptides. This is in effect an antagonistic activity which will enhance the immunogenicity of vaccines, more specifically vaccines that that contain the proteins with the aforementioned immune suppressive activity.

In another aspect, the current invention concerns the monomeric form of any immune suppressive peptide sequence which shows immune suppressive activity as dimer or multimer or when coupled to a carrier protein, is useful as an adjuvant.

In another aspect, the current invention concerns peptides encompassing immune suppressive domains and containing small alterations (mutations, post translational modifications, Chemical alterations of the amino acid residues in such peptides, insertions or deletions of amino acid residues) will result in peptides that bind to but will not activate the cellular machinery that produces immune suppression. Such altered immune suppressive domain peptides will function as agents that will enhance the immune responses to molecules that contain the aforementioned immune suppressive activity and can be used as adjuvants.

In yet another aspect of the current invention, small molecules antagonists of the cellular interaction partners of the immune suppressive domain peptides, will enhance immune responses to vaccines.

Certain aspects of the invention are provided in the claims.

According to an aspect, the invention concerns an adjuvant comprising an antagonist to an immune suppressive domain or a mutated immune suppressive domain.

According to an aspect, the invention concerns an adjuvant comprising a peptide, said peptide comprising an immune suppressive domain or a mutated immune suppressive domain.

According to an aspect, the invention concerns an adjuvant comprising a peptide, which is a monomeric peptide, having a dimer or trimer or multimer, which exhibits immune suppressive activity; or wherein said peptide is a mutated form of said monomeric peptide.

According to an aspect, the invention concerns an immunosuppressive domain selected among the immunosuppressive domains of Table 1 and the sequences of the present invention.

According to an aspect, the invention concerns the use of an immunosuppressive domain as an adjuvant.

According to an aspect, the invention concerns a monomeric peptide, having a dimer, which shows immune suppressive activity.

According to an aspect, the invention concerns a biological entity selected among an adjuvant according to the invention, an immunosuppressive domain according to the invention, and a monomeric peptide according to the invention.

According to an aspect, the invention concerns a vaccine composition comprising a biological entity of the invention and a vaccine antigen.

According to an aspect, the invention concerns a kit-of-parts comprising a vaccine composition of the invention and a second active ingredient.

According to an aspect, the invention concerns a method of treating, preventing or ameliorating a clinical condition, said method comprising administering a biological entity of the invention or a vaccine composition of the invention.

According to an aspect, the invention concerns the use of a biological entity of the invention for the manufacture of a medicament for the treatment, amelioration or prevention of a clinical condition, such as a viral infection.

According to an aspect, the invention concerns a biological entity of the invention for treating, ameliorating or preventing a clinical condition, such as a viral infection.

According to an aspect, the invention concerns a pharmaceutical composition comprising a biological entity of the invention.

According to an aspect, the invention concerns a method of reducing the risk of an individual encountering a clinical condition, said method comprising administering a biological entity of the invention to the individual in an amount sufficient to generate a protective immune response.

According to an aspect, the invention concerns a method of producing a vaccine composition, comprising combining:
a. A vaccine antigen; and
b. An adjuvant of the invention.

According to an aspect, the invention concerns a vaccine comprising at least one biological entity of the invention.

According to an aspect, the invention concerns a treatment of infected individuals using at least one biological entity of the invention.

According to an aspect, the invention concerns a prophylactic treatment of individuals suffering from an infection using a biological entity of the invention.

According to an aspect, the invention concerns a vaccination modality comprising at least one biological entity of the invention.

According to an aspect, the invention concerns a vaccine comprising an immune suppressive domain of the invention, such as of Table 1.

According to an aspect, the invention concerns an immune suppressive domain of the invention, wherein said immune suppressive domain have abrogated immunosuppressive properties for use in a vaccine.

According to an aspect, the invention concerns a peptide derived from an immunosuppressive domain selected among seqid 209 to seqid 281, and the sequences of Table 1; by performing 1, 2, 3, 4, or more mutations, insertions or deletions.

According to an aspect, the invention concerns a vaccine comprising a mutated immunosuppressive domain selected among seqid 209 to seqid 281 and the peptides of the invention, wherein the immunosuppressive properties of said domain have been reduced or abrogated.

### DETAILED DISCLOSURE

The present invention further concerns a number of embodiments. Certain embodiments are provided in the claims.

According to an embodiment, the invention concerns an adjuvant comprising an antagonist to an immune suppressive domain or a mutated immune suppressive domain.

According to an embodiment, the invention concerns an adjuvant comprising a peptide, said peptide comprising an immune suppressive domain or a mutated immune suppressive domain.

An immune suppressive peptide is a peptide that can inhibit proliferation of CTLL-2 or PBMCs in assays, as described in the examples, by more than 20%.

According to an embodiment, the invention concerns the adjuvant, wherein said mutated immune suppressive domain comprise 1, 2, 3 or 4 mutations, deletions or insertions with respect to the non-mutated form.

The term “mutation” is used with a number about this number of point mutation(s), i.e. 3 mutations mean 3 point mutations. The term “deletion” is used with a number about the deletion of this number of amino acid(s), i.e. 2 deletions means the deletion of 2 amino acids. The term “insertion” is used with a number about insertion of this number of amino acid(s), i.e. 1 insertion means the insertion of 1 amino acid.

According to an embodiment, the invention concerns an adjuvant comprising a peptide, which is a monomeric peptide, having a dimer or trimer or multimer, which exhibits immune suppressive activity; or wherein said peptide is a mutated form of said monomeric peptide.

According to an embodiment, the invention concerns an adjuvant of the invention, wherein said mutated form comprise 1, 2, 3 or 4 mutations, deletions or insertions with respect to the non-mutated form.

According to an embodiment, the invention concerns the adjuvant of the invention, wherein said peptide forms part of the surface protein of a pathogen, such as a virus.

According to an embodiment, the invention concerns the adjuvant, wherein said peptide forms part of the surface protein of a virus.

According to an embodiment, the invention concerns the adjuvant, wherein said peptide forms part of an enveloped virus surface glycoprotein.

According to an embodiment, the invention concerns the adjuvant, wherein said peptide has a length of at least 8, preferably 9, more preferred 10, preferably 11, more preferred 12, preferably 13, more preferred 14, preferably 15, more preferred 16, preferably 17, more preferred 18 amino acids.

According to an embodiment, the invention concerns the adjuvant, wherein said peptide has a length selected among 5-200, preferably 10-100, more preferred 20-50, preferably 30-40 amino acids.

According to an embodiment, the invention concerns the adjuvant, further comprising a fusion peptide from a fusion protein.

According to an embodiment, the invention concerns the adjuvant, comprising a fusion peptide from the fusion protein of an enveloped virus.

According to an embodiment, the invention concerns the adjuvant, comprising a fusion peptide from a type I fusion protein.

According to an embodiment, the invention concerns the adjuvant, comprising a fusion peptide from a type II fusion protein.

According to an embodiment, the invention concerns the adjuvant, in which said fusion peptide has 1, 2, 3 or 4 mutations, deletions or insertions with respect to the wild type.

According to an embodiment, the invention concerns the adjuvant, wherein said peptide, or a functional homologue thereof, binds to the STING complex.

According to an embodiment, the invention concerns the adjuvant, wherein said peptide, or a functional homologue thereof, affects type I interferon responses.

According to an embodiment, the invention concerns the adjuvant, wherein said peptide, or a functional homologue thereof, affects type I interferon responses induced by membrane fusion.

According to an embodiment, the invention concerns the adjuvant, comprising a peptide from Table 1 or a peptide selected among the sequences 1 to 281.

According to an embodiment, the invention concerns the adjuvant, comprising a peptide with seq id 275.

According to an embodiment, the invention concerns the adjuvant in which said peptide has immune suppressive activity as dimer or multimer or when coupled to carrier proteins.

By immune suppressive activity is meant that it can inhibit proliferation of CTLL-2 or PBMCs in assays as described in the examples, by more than 20%, preferably by more than 30%, more preferred by more than 50%.

According to an embodiment, the invention concerns the adjuvant in which said peptide has no or diminished immune suppressive activity as a monomer while having immune suppressive activity in the dimeric form.

No or diminished immune suppressive activity means that the immune suppressive activity is suppressed less than 20%.

According to an embodiment, the invention concerns the adjuvant in which said peptide contains at least one non-genetically encoded amino acid residue.

According to an embodiment, the invention concerns the adjuvant in which said peptide contains at least one D-amino acid.

According to an embodiment, the invention concerns the adjuvant in which said peptide contains at least one D-amino acid residue.

According to an embodiment, the invention concerns the adjuvant in which said peptide is coupled to any other molecule.

The molecule may e.g. be a ligand of a receptor, thereby targeting the peptide, or it may e.g. be a molecule providing different solubility characteristics of the combination of the peptide and the molecule as compared to the peptide alone, or the molecule may be a nanoparticle. The peptide may further form part of a protein, which may provide advantages such as easy production, as the protein may be derived from natural sources.

According to an embodiment, the invention concerns the adjuvant in which said peptide is attached to at least one lipid.

According to an embodiment, the invention concerns the adjuvant in which said peptide is coupled to a molecule through a peptide bond.

According to an embodiment, the invention concerns the adjuvant in which said peptide is coupled to a protein.

According to an embodiment, the invention concerns the adjuvant in which said peptide is a circular peptide.

According to an embodiment, the invention concerns the adjuvant in which said peptide is attached to at least one biological membrane.

According to an embodiment, the invention concerns the adjuvant in which said peptide is modified in a way in which one of the peptide bonds is replaced by a non-peptide bond.

According to an embodiment, the invention concerns the adjuvant comprising a functional homologue of any peptide according to the invention.

According to an embodiment, the invention concerns the adjuvant comprising an antagonist of a peptide according to the invention.

According to an embodiment, the invention concerns an immunosuppressive domain selected among the immunosuppressive domains of Table 1 and the sequences.

According to an embodiment, the invention concerns a use of an immunosuppressive domain as an adjuvant.

According to an embodiment, the invention concerns said use, wherein said immunosuppressive domain is from a virus.

According to an embodiment, the invention concerns said use, wherein said immunosuppressive domain is from an influenza virus.

According to an embodiment, the invention concerns said use, wherein said adjuvant is for a vaccine for the treatment or prophylaxis of a virus infection.

According to an embodiment, the invention concerns said use, wherein said virus infection and said immunosuppressive domain is from the same genus of virus.

According to an embodiment, the invention concerns said use, wherein said virus infection and said immunosuppressive domain is from the same species of virus.

According to an embodiment, the invention concerns said use, wherein said virus infection is an influenza virus.

According to an embodiment, the invention concerns a monomeric peptide, having a dimer, which shows immune suppressive activity.

According to an embodiment, the invention concerns a biological entity selected among an adjuvant according to the invention, an immunosuppressive domain according to the invention, and a monomeric peptide according to the invention.

According to an embodiment, the invention concerns a vaccine composition comprising a biological entity according to the invention and a vaccine antigen.

According to an embodiment, the invention concerns a vaccine composition for influenza, comprising an influenza antigen and a peptide which forms part of an immunosuppressive domain of an influenza.

According to an embodiment, the invention concerns a vaccine composition, wherein said antigen and said immunosuppressive domain is from the same clade or strain of influenza.

According to an embodiment, the invention concerns a kit-of-parts comprising the vaccine composition according to the invention and a second active ingredient.

According to an embodiment, the invention concerns a method of treating, preventing or ameliorating a clinical condition, said method comprising administering a biological entity according to the invention or a vaccine composition according to the invention.

According to an embodiment, the invention concerns a use of a biological entity according to the invention for the manufacture of a medicament for the treatment, amelioration or prevention of a clinical condition, such as a viral infection. The viral infection may preferably be a viral infection of Table 1.

According to an embodiment, the invention concerns the biological entity of the invention for treating, ameliorating or preventing a clinical condition, such as a viral infection.

According to an embodiment, the invention concerns a pharmaceutical composition comprising a biological entity according to the invention.

According to an embodiment, the invention concerns a method of reducing the risk of an individual encountering a clinical condition, said method comprising administering a biological entity according to the invention, to the individual in an amount sufficient to generate a protective immune response.

According to an embodiment, the invention concerns a method of producing the vaccine composition of the invention, comprising combining:
a. A vaccine antigen; and
b. An adjuvant of the invention.

According to an embodiment, the invention concerns a vaccine comprising at least one biological entity of the invention.

According to an embodiment, the invention concerns a treatment of infected individuals using at least one biological entity according to the invention.

According to an embodiment, the invention concerns a prophylactic treatment of individuals infection using a biological entity of the invention.

According to an embodiment, the invention concerns a vaccination modality comprising at least one biological entity of the invention.

According to an embodiment, the invention concerns a vaccine comprising an immune suppressive domain of the invention or Table 1.

According to an embodiment, the invention concerns the immune suppressive domain according to the invention, wherein said immune suppressive domain have abrogated immunosuppressive properties for use in a vaccine

According to an embodiment, the invention concerns a peptide derived from an immunosuppressive domain selected among seqid 209 to seqid 281, and the sequences of Table 1; by performing 1, 2, 3, 4, or more mutations, insertions or deletions.

According to an embodiment, the invention concerns a vaccine comprising a mutated immunosuppressive domain according to seqid 209 to seqid 281 and the peptides of the invention, wherein the immunosuppressive properties of said domain have been reduced or abrogated.

The co-pending patent application PCT/DK2012/050381 as well as Table 1 provides a number of immunosuppressive domains.

All cited references are incorporated by reference.

The accompanying Figures and Examples are provided to explain rather than limit the present invention. It will be clear to the person skilled in the art that aspects, embodiments and claims of the present invention may be combined.

TABLE 1

| | | | | Putative ISU as identified |
|---|---|---|---|---|
| | | | | using the criteria described |
| | | | | in this application for |
| | | Species | Species | identification of |
| Family | Genus | (group) | (Strain) | immunosuppressive domains |
| Flavi- | Flavi- | Aroa virus | Bussuquara virus | seqid85 |
| viridae | virus | | guape virus | NRGWNNGCGLFGKG |
| | | | Naranjal virus | ************** |
| | | | | seqid7 |
| | | | | GDAAWDFGSVGGVFNSLGK |
| | | | | **o****o*****oo*o** |
| | | Dengue virus | Dengue 1 | seqid8 |
| | | | | GGTAWDFGSIGGVFTSVGK |
| | | | | *o***************** |
| | | | Dengue 2 | seqid9 |
| | | | | GDTAWDFGSLGGVFTSVGK |
| | | | | ****************o** |
| | | | | seqid173 |
| | | | | KGSSIGKMFEATARGARRMAILG |
| | | | Dengue 3 | seqid174 |
| | | | | KGSSIGQMFETTMRGAKRMAILG |
| | | | Dengue 4 | seqid10 |
| | | | | GETAWDFGSVGGLLTSLGK |
| | | | | ************oo***** |
| | | | | seqid173 |
| | | | | KGSSIGKMFEATARGARRMAILG |
| | | Japanese | Japanese encephalitis virus | seqid11 |
| | | encephalitis | | LGDTAWDFGSIGGVFNSIG |
| | | virus group | | ***o*************** |
| | | | Koutango virus | seqid12 |
| | | | | LGDTAWDFGSVGGIFTSLG |
| | | | Murray Valley encephalitis virus | seqid13 |
| | | | | LGDTAWDFGSVGGVFNSIG |
| | | | St. Louis encephalitis virus | seqid11 |
| | | | | LGDTAWDFGSIGGVFNSIG |
| | | | | ******************* |
| | | | Usutu virus | seqid14 |
| | | | | LGDTAWDFGSVGGIFNSVG |
| | | | | ******************* |
| | | | West Nile virus | seqid15 |
| | | | | LGDTAWDFGSVGGVFTSVG |
| | | | | **********o******** |
| | | Kokobera virus | Kokobera virus unclassified Kokobera | seqid16 |
| | | group | virus group | IGDDAWDFGSVGGILNSVG |
| | | Modoc virus | Modoc virus | seqid17 |
| | | group | | VGSAFWNSDQRFSAINLMD |
| | | | | seqid18 |
| | | | | DRGWGNGCALFGKG |
| | | | Cowbone Ridge virus | |
| | | | Jutiapa virus | |
| | | | Sal Vieja virus | |
| | | | San Perlita virus | |
| | | mosquito-borne | Ilheus virus | seqid84 |
| | | viruses | | LGDTAWDFGSVGGIFNSIG |
| | | | Sepik virus | seqid19 |
| | | | | TGEHSWDFGSTGGFFASVG |
| | | Ntaya virus | Bagaza virus | seqid20 |
| | | group | | LGDTAWDFGSVGGFFTSLG |
| | | | Tembusu virus | seqid83 |
| | | | | LGDTAWDFGSVGGVLTSIG |
| | | | Yokose virus | seqid21 |
| | | | | IGDDAWDFGSTGGIFNTIG |
| | | Rio Bravo virus | Apoi virus | seqid22 |
| | | group | | SSAFWNSDEPFHFSNLISII |
| | | | Entebbe bat virus | seqid23 |
| | | | | GDDAWDFGSTGGIFNTIGKA |
| | | | Rio Bravo virus | seqid24 |
| | | | | SSAYWSSSEPFTSAGIMRIL |
| | | | Saboya virus | seqid18 |
| | | | | DRGWGNGCALFGKG |
| | | | | seqid25 |
| | | | | GSSSWDFSSAGGFFGSIGKA |
| | | Seaborne tick- | Meaban virus | seqid26 |
| | | borne virus | | GDAAWDFGSVGGFMTSIGRA |
| | | group | | seqid27 |
| | | | | DRGWGNHCGLFGKG |
| | | | Saumarez Reef virus | seqid28 |
| | | | | GETAWDFGSAGGFFTSVGRG |
| | | | | seqid27 |
| | | | | DRGWGNHCGLFGKG |
| | | | Tyuleniy virus | seqid29 |
| | | | | GEAAWDFGSAGGFFQSVGRG |
| | | | | seqid27 |
| | | | | DRGWGNHCGLFGKG |
| | | Spondweni virus | Zika virus | seqid30 |
| | | group | | LGDTAWDFGSVGGVFNSLGK |
| | | | | *************oo**o** |
| | | | Kyasanur forest disease virus | seqid31 |
| | | | | VGEHAWDFGSVGGMLSSVG |
| | | | | *************o***** |
| | | | | seqid27 |
| | | | | DRGWGNHCGLFGKG |
| | | | Langat virus | seqid32 |
| | | | | VLGEHAWDFGSVGGVMTSIG |
| | | | | seqid27 |
| | | | | DRGWGNHCGLFGKG |
| | | | Louping ill virus | seqid33 |
| | | | | IGEHAWDFGSAGGFFSSIG |
| | | | | **********o***oo*o* |
| | | | | seqid27 |
| | | | | DRGWGNHCGLFGKG |
| | | | Omsk hemorrhagic fever virus | seqid34 |
| | | | | LGEHAWDFGSTGGFLSSIG |
| | | | | seqid27 |
| | | | | DRGWGNHCGLFGKG |
| | | | Powassan virus | seqid35 |
| | | | | VGEHAWDFGSVGGILSSVG |
| | | | | *************o***** |
| | | | | seqid36 |
| | | | | DRGWGNHCGFFGKG |
| | | | | ************* |
| | | | Royal Farm virus | seqid27 |
| | | | | DRGWGNHCGLFGKG |
| | | | Tick-borne encephalitis virus | seqid37 |
| | | | | IGEHAWDFGSAGGFLSSIG |
| | | | | seqid38 |
| | | | | IGEHAWDFGSTGGFLTSVG |
| | | | | seqid39 |
| | | | | IGEHAWDFGSTGGFLASVG |
| | | | | seqid27 |
| | | | | DRGWGNHCGLFGKG |
| | | Yaounde virus | | seqid40 |
| | | | | LGDTAWDFGSIGGVFTSLG |
| | | Yellow fever | Banzi virus | seqid41 |
| | | virus group | | VGSSSWDFSSTSGFFSSVG |
| | | | Bouboui virus | seqid42 |
| | | | | VGRSSWDFSSAGGFFSSVG |
| | | | Edge Hill virus | |
| | | | Uganda S virus | |
| | | | Wesselsbron virus | |
| | | | Yellow fever virus | seqid43 |
| | | | | MGDTAWDFSSAGGFFTSVG |
| | | | | ***o*************** |
| | | unclassified | Batu Cave virus | seqid44 |
| | | Flavivirus | Cacipacore virus | NRGWGTGCFKWGIG |
| | | | Calbertado virus | seqid45 |
| | | | Cell fusing agent virus | NRGWGTGCFEWGLG |
| | | | Chaoyang virus | |
| | | | Chimeric Tick-borne encephalitis | |
| | | | virus/Dengue virus 4 | |
| | | | Culex theileri flavivirus | |
| | | | Donggang virus | |
| | | | Duck hemorrhagic ovaritis virus | |
| | | | Flavivirus Aedes/MO-Ac/ITA/2009 | |
| | | | Flavivirus Anopheles/PV-Am/ITA/2009 | |
| | | | Flavivirus CbaAr4001 | |
| | | | Flavivirus FSME | |
| | | | Flavivirus | |
| | | | Phlebotomine/76/Arrabida/2007 | |
| | | | Gadgets Gully virus | |
| | | | Greek goat encephalitis virus | |
| | | | Jugra virus | |
| | | | Kadam virus | |
| | | | Kamiti River virus | |
| | | | Kedougou virus | |
| | | | Montana myotis leukoencephalitis | |
| | | | virus | |
| | | | Mosquito flavivirus | |
| | | | Ngoye virus | |
| | | | Nounane virus | |
| | | | Phlebotomus flavivirus Alg_F19 | |
| | | | Phlebotomus flavivirus Alg_F8 | |
| | | | Quang Binh virus | |
| | | | Russian Spring-Summer encephalitis | |
| | | | virus | |
| | | | Sokoluk virus | |
| | | | Spanish sheep encephalitis virus | |
| | | | T′Ho virus | |
| | | | Tai forest virus B31 | |
| | | | Tamana bat virus | |
| | | | Tick-borne flavivirus | |
| | | | Wang Thong virus | |
| | | | Flavivirus sp. | |
| | | | Aedes flavivirus | seqid45 |
| | | | | NRGWGTGCFEWGLG |
| | | | | seqid46 |
| | | | | HVAGRYSKHGMAGIGSVWEDLVR |
| | | | Culex flavivirus | seqid44 |
| | | | | NRGWGTGCFKWGIG |
| | | | | seqid47 |
| | | | | VDKYRRFGTAGVGG |
| | Hepaci- | Hepatitis C | Hepatitis C virus genotype 1 a | |
| | virus | virus | | |
| | | | Hepatitis C virus genotype lb | seqid48 |
| | | | | GLIHLHRNIVDVQYLYG |
| | | | | seqid176 |
| | | | | PALSTGLIHLHRNIVDVQ |
| | | | Hepatitis C virus genotype 2 | seqid49 |
| | | | | GLIHLHQNIVDVQYMYG |
| | | | | seqid175 |
| | | | | PALSTGLIHLHQNIVDVQ |
| | | | Hepatitis C virus genotype 3 | seqid175 |
| | | | | PALSTGLIHLHQNIVDVQ |
| | | | Hepatitis C virus genotype 4 | seqid175 |
| | | | | PALSTGLIHLHQNIVDVQ |
| | | | Hepatitis C virus genotype 5 | seqid50 |
| | | | | GLIHLHQNIVDTQYLYG |
| | | | | seqid177 |
| | | | | PALSTGLIHLHQNIVDTQ |
| | | | Hepatitis C virus genotype 6 | seqid175 |
| | | | | PALSTGLIHLHQNIVDVQ |
| | | | All Hepatitis C virus | |
| | Pesti virus | Border disease | Border disease virus - | seqid51 |
| | | virus | Border disease virus - X818 | NTTLLNGSAFQLICPYGWVGRVEC |
| | | | Border disease virus 1 | seqid52 |
| | | | Border disease virus 2 | SYFQQYMLKGQYQYWFDLE |
| | | | Border disease virus 3 | |
| | | | Border disease virus isolates | |
| | | Bovine viral | Bovine viral diarrhea virus 1-CP7 | seqid53 |
| | | diarrhea virus 1 | Bovine viral diarrhea virus 1-NADL | NTTLLNGPAFQMVCPLGWTGTVSC |
| | | | Bovine viral diarrhea virus 1-Osloss | seqid54 |
| | | | Bovine viral diarrhea virus 1-SD1 | SYFQQYMLKGEYQYWFDLE |
| | | | Bovine viral diarrhea virus isolates | |
| | | | and strains | |
| | | | Bovine viral diarrhea virus type 1a | |
| | | | Bovine viral diarrhea virus type 1b | |
| | | | Pestivirus isolate 97-360 | |
| | | | Pestivirus isolate Hay 87/2210 | |
| | | | Pestivirus strain mousedeer | |
| | | | Pestivirus type 1 isolates | |
| | | Bovine viral | Bovine viral diarrhea virus 2 | seqid55 |
| | | diarrhea virus 2 | Pestivirus sp. strain 178003 | SLLNGPAFQMVCPQGWTGTIEC |
| | | (BVDV-2) | Pestivirus sp. strain 5250Giessen-3 | seqid56 |
| | | | Bovine viral diarrhea virus-2 isolate | DRYFQQYMLKGKWQYWFDLD |
| | | | SCP | |
| | | Classical swine | Classical swine fever virus | seqid57 |
| | | fever virus | Hog cholera virus strain Zoelen | TLLNGSAFYLVCPIGWTGVIEC |
| | | | | seqid58 |
| | | | | SYFQQYMLKGEYQYWFDLD |
| | | unclassified | Bovine viral diarrhea virus 3 | seqid59 |
| | | Pestivirus | | TLLNGPAFQLVCPYGWTGTIEC |
| | | | | seqid60 |
| | | | | DNYFQQYMLKGKYQYWFDLEATD |
| | | | Chamois pestivirus 1 | seqid61 |
| | | | | TLLNGSAFQMVCPFGWTGQVEC |
| | | | | seqid62 |
| | | | | DSYFQQYMLKGEYQYWFDLDAKD |
| | | | Porcine pestivirus isolate | seqid205 |
| | | | Bungowannah | TLLNGPAFQLVCPYGWTGTIECDSYYQQ |
| | | | | YIIKSGYQYWFDLTAKD |
| | Unnclassified | | Barkedji virus | |
| | Flaviviridae | | Canine hepacivirus AAK-2011 | |
| | | | GB virus A | |
| | | | Douroucouli hepatitis GB virus A | |
| | | | GBV-A-like agents | |
| | | | GB virus D | |
| | | | GBV-C/HGV group | |
| | | | GB virus C | |
| | | | Hepatitis GB virus C-like virus | |
| | | | Hepatitis GB virus B | |
| | | | Lammi virus | |
| | | | Marmoset hepatitis GB virus A | |
| | | | Nakiwogo virus | |
| | | | Turkey meningoencephalitis virus | |
| Togaviridae | Alpha-virus | Aura virus | | seqid63 |
| | | Barmah Forest | | GVYPFMWGGAYCFCDTENTQVS |
| | | virus | | **********o****o**o*o* |
| | | Middelburg virus | | seqid64 |
| | | Ndumu virus | | APFGCEIYTNPIRAENCAVGSIP |
| | | Salmon pancreas | | *****o*ooo*o**oo*oo*oo* |
| | | disease virus | | seqid65 |
| | | Getah virus | | SDFGGIATVKYSASKSGKCAVH |
| | | Mayaro virus | | o***oooooo*ooooo*o*oo* |
| | | Trocara virus | | seqid66 |
| | | EEEV complex | | FSTANIHPEFRLQICTSYVTCKGDCHPP |
| | | | | *oooooooo*oooo*ooooo*ooo*o** |
| | | WEEV complex | Fort Morgan virus | |
| | | | Highlands J virus | |
| | | | Sindbis virus | |
| | | | Western equine encephalomyelitis | |
| | | | virus | |
| | | | Whataroa virus | |
| | | VEEV complex | Cabassou virus | |
| | | | Mucambo virus | |
| | | | Pixuna virus | |
| | | | Venezuelan equine encephalitis virus | seqid67 |
| | | | | GVYPFMWGGAYCFCD |
| | | | | *************** |
| | | | | seqid68 |
| | | | | GDCHPPKDHIVTHPQYHAQ |
| | | | | ************o**o*o* |
| | | | | seqid69 |
| | | | | AVSKTAWTWLTS |
| | | | | *********oo* |
| | | SFV complex | Bebaru virus | seqid63 |
| | | | O′nyong-nyong virus | GVYPFMWGGAYCFCDTENTQVS |
| | | | Ross River virus | **********o****o**o*o* |
| | | | Semliki forest virus | seqid64 |
| | | | Una virus | APFGCEIYTNPIRAENCAVGSIP |
| | | | | *****o*ooo*o**oo*oo*oo* |
| | | | | seqid65 |
| | | | | SDFGGIATVKYSASKSGKCAVH |
| | | | | o***oooooo*ooooo*o*oo* |
| | | | | seqid66 |
| | | | | FSTANIHPEFRLQICTSYVTCKGDCHPP |
| | | | | *oooooooo*oooo*ooooo*ooo*o** |
| | | | Chikungunya virus | seqid67 |
| | | | | GVYPFMWGGAYCFCD |
| | | | | *************** |
| | | | | seqid70 |
| | | | | VHCAAECHPPKDHIVNY |
| | | | | oo*o*o**o******** |
| | | | | seqid71 |
| | | | | PASHTTLGVQDISATAMSWV |
| | | | | o****oo******o****** |
| | Rubivirus | Rubellavirus | Rubella virus(strain BRD1) | seqid72 |
| | | | Rubella virus(strain BRDII) | ACTFWAVNAYSSGGYAQLASYFNPGGSYYK |
| | | | Rubella virus(strain Cendehill) | ***o*o****o**oo****o**o******o |
| | | | Rubella virus(strain M33) | seqid73 |
| | | | Rubella virus(strain RN-UK86) | QYHPTACEVEPAFGHSDAACWGFPTDT |
| | | | Rubella virus(strain THERIEN) | ***o*o*o*o****o********o*** |
| | | | Rubella virus(strain TO-336 vaccine) | seqid74 |
| | | | Rubella virus(strain TO-336) | MSVFALASYVQHPHKTVRVKFHT |
| | | | Rubella virus(vaccine strain RA27/3) | ***oo*****o**o**o****** |
| | | | | seqid159 |
| | | | | ETRTVWQLSVAGVSC |
| | | | | o*o*********oo* |
| | | | | seqid76 |
| | | | | NVTTEHPFCNMPHGQLEVQVPP |
| | | | | o*o*o**oo*o*o****o*oo* |
| | | | | seqid77 |
| | | | | DPGDLVEYIMNYTGNQQSRW |
| | | | | ****o******o*o****** |
| | | | | seqid78 |
| | | | | GSPNCHGPDWASPVCQRHSPDCS |
| | | | | ****o***o************** |
| | | | | seqid79 |
| | | | | RLVGAT P E RP RL RLV |
| | | | | o***o**o**o**** |
| | | | | seqid80 |
| | | | | DADDPLLRTAPGP |
| | | | | *oo********** |
| | | | | seqid81 |
| | | | | GEVWVTPVIGSQARKCGL |
| | | | | oo*o**o**o*****o** |
| | | | | seqid86 |
| | | | | HIRAGPYGHATVEM |
| | | | | oo***********o |
| | | | | seqid87 |
| | | | | PEWIHAHTTSDPWHP |
| | | | | o**oooo*o***o*o |
| | | | | seqid88 |
| | | | | PGPLGLKFKTVRPVALPR |
| | | | | ****o***o**o*oo*** |
| | | | | seqid89 |
| | | | | ALAPPRNVRVTGCYQCGTPAL |
| | | | | oooo**o*o*o**o******* |
| | | | | seqid90 |
| | | | | EGLAPGGGNCHLTVNGEDVG |
| | | | | ***o*****o**oo*o*oo* |
| | | | | seqid207 |
| | | | | LLNTPPPYQVSCGG |
| | | | | ******o*o*o*** |
| | | | | seqid92 |
| | | | | RASARVIDPAAQSFTGVVYGTHT |
| | | | | **o***oo*o************* |
| Bunya- | Hanta-virus | Amur virus | | seqid93 |
| viridae | (continued on | Bayou virus | | TAVSETRQTWAEWAAAHWWQLTLG |
| | next page) | Black Creek | | *******ooo*****o******* |
| | | Canal virus | | seqid94 |
| | | Cano Delgadito | | NPPDCPGVGTGCTACGVYLD |
| | | virus | | **o****o********o*** |
| | | Calabazo virus | | seqid95 |
| | | Catacamas virus | | RKVCIQLGTEQTCKTIDSNDC |
| | | Choclo virus | | *oo*o*o*o*oo**oo*o*** |
| | | Dobrava-Belgrade | | seqid96 |
| | | virus | | DTLLFLGPLEEGGMIFKQWCTTTCQFGD |
| | | El Moro Canyon | | PGDIM |
| | | virus | | seqid97 |
| | | Hantaan virus | | GSFRKKCSFATLPSCQYDGNTVSG |
| | | Isla Vista virus | | *o***o*o***o*o*ooo**oo** |
| | | Khabarovsk virus | | seqid98 |
| | | Laguna Negra | | ATKDSFQSFNITEPH |
| | | virus | | **o****o**oooo* |
| | | Limestone Canyon | | seqid99 |
| | | virus | | GSGVGFNLVCSVSLTEC |
| | | Monongahela | | ******o*o*ooo**** |
| | | virus | | seqid100 |
| | | Muleshoe virus | | KACDSAMCYGSSTANLVRGQNT |
| | | Muju virus | | ****o*o***ooooo*o**o** |
| | | New York virus | | seqid101 |
| | | Oran virus | | GKGGHSGSKFMCCHDKKCSATGLVAAAP |
| | | Playa de Oro | | HL |
| | | virus | | ********o*o***ooo*ooo**o*oo* |
| | | Prospect Hill | | ** |
| | | virus | | seqid102 |
| | | Puumala virus | | DDGAPQCGVHCWFKKSGEW |
| | | Rio Mamore virus | | ***o*o*ooo***oo**** |
| | | Rio Segundo | | |
| | | virus | | |
| | | Saaremaa virus | | |
| | | Seoul virus | | |
| | | Sin Nombre virus | | |
| | | Soochong virus | | |
| | | Thailand virus | | |
| | | Thottapalayam | | |
| | | virus | | |
| | | Topografov virus | | |
| | | Tula virus | | |
| | Ortho-bunya- | Anopheles A | | seqid103 |
| | virus | virus | | KHDELCTGPCPVNINHQTGWLT |
| | | Anopheles B | | *o*o***o**oooooooo*o*o |
| | | virus | | seqid104 |
| | | Bakau virus | | WGCEEFGCLAVSDGCVFGSCQD |
| | | Batama virus | | **o*oo**o*ooo**oo***** |
| | | Bwamba virus | | seqid105 |
| | | Caraparu virus | | GNGVPRFDYLCHLASRKEVIVRKC |
| | | Kaeng Khoi virus | | *o*ooo*ooo*oooo*ooooo*o* |
| | | Kairi virus | | seqid106 |
| | | Madrid virus | | SCAGCINCFQNIHC |
| | | Main Drain virus | | *o**ooooooooo* |
| | | Marituba virus | | |
| | | Nyando virus | | |
| | | Oriboca virus | | |
| | | Oropouche virus | | |
| | | Sathuperi virus | | |
| | | Shamonda virus | | |
| | | Shuni virus | | |
| | | Simbu virus | | |
| | | Tacaiuma virus | | |
| | | Tete virus | | |
| | | Turlock virus | | |
| | | unclassified | | |
| | | Orthobunyavirus | | |
| | | Akabane virus | Sabo virus | |
| | | | Tinaroo virus | |
| | | | Yaba-7 virus | |
| | | Bunyamwera | Batai virus | |
| | | virus | Birao virus | |
| | | | Bozo virus | |
| | | | Cache Valley virus | |
| | | | Fort Sherman virus | |
| | | | Germiston virus | |
| | | | Guaroa virus | |
| | | | Iaco virus | |
| | | | Ilesha virus | |
| | | | Lokern virus | |
| | | | Maguari virus | |
| | | | Mboke virus | |
| | | | Ngari virus | |
| | | | Northway virus | |
| | | | Playas virus | |
| | | | Potosi virus | |
| | | | Shokwe virus | |
| | | | Tensaw virus | |
| | | | Tlacotalpan virus | |
| | | | Xingu virus | |
| | | California | California encephalitis serogroup | |
| | | Encephalitis | virus LEIV | |
| | | virus | California encephalitis virus - BFS- | |
| | | | 283 | |
| | | | Chatanga virus | |
| | | | Inkoo virus | |
| | | | Jamestown Canyon virus | |
| | | | Jamestown Canyon-like virus | |
| | | | Jerry Slough virus | |
| | | | Keystone virus | |
| | | | La Crosse virus | |
| | | | Lumbo virus | |
| | | | Melao virus | |
| | | | Morro Bay virus | |
| | | | San Angelo virus | |
| | | | Serra do Navio virus | |
| | | | Snowshoe hare virus | |
| | | | South River virus | |
| | | | Tahyna virus | |
| | | | Trivittatus virus | |
| | | Caraparu virus | Apeu virus | |
| | | | Bruconha virus | |
| | | | Ossa virus | |
| | | | Vinces virus | |
| | | Manzanilla virus | Buttonwillow virus | |
| | | | Ingwavuma virus | |
| | | | Mermet virus | |
| | | Marituba virus | Gumbo Limbo virus | |
| | | | Murutucu virus | |
| | | | Nepuyo virus | |
| | | | Restan virus | |
| | | Wyeomyia virus | Anhembi virus | |
| | | | BeAr328208 virus | |
| | | | Macaua virus | |
| | | | Sororoca virus | |
| | | | Taiassui virus | |
| | Phlebovirus | Bujaru virus | | |
| | | Candiruvirus | | |
| | | Chilibre virus | | |
| | | Frijoles virus | | |
| | | Punta | | |
| | | Tor [symbol] Salehabad | | |
| | | virus | | |
| | | Sandflyfever | | |
| | | Naples virus | | |
| | | Uukuniemi viruso | | |
| | | virus | | |
| | | Rift Valley | | seqid107 |
| | | fever virus | | KTVSSELSCREGQSYWT |
| | | | | **oo**oo*o**o*o** |
| | | | | seqid108 |
| | | | | GSFSPKCLSSRRC |
| | | | | *******oooooo |
| | | | | seqid109 |
| | | | | ENKCFEQCGGWGCGCFNVNPSCLFVHT |
| | | | | **o*o**o*oo*oo***ooo***o**o |
| | | | | seqid110 |
| | | | | WGSVSLSLDAEGISGSNSFSF |
| | | | | **ooo*o**o*o*o*o*oo** |
| | | | | seqid111 |
| | | | | RQGFLGEIRCNSE |
| | | | | *o*****o**oo* |
| | | | | seqid112 |
| | | | | AHESCLRAPNLVSYKPMIDQLEC |
| | | | | *oo**oo**oooo*o*oo*ooo* |
| | | | | seqid113 |
| | | | | DPFVVFERGSLPQTR |
| | | | | **ooo*oo*o***o* |
| | | | | seqid114 |
| | | | | QAFSKGSVQADLTLMFD |
| | | | | **ooo*ooo*oooooo* |
| | | | | seqid115 |
| | | | | CDAAFLNLTGCYSCNAG |
| | | | | *o*o*o*oo*****oo* |
| | | | | seqid116 |
| | | | | CQILHFTVPEVEEEEMYSC |
| | | | | *ooo*ooo*ooooooo*o* |
| | | | | seqid117 |
| | | | | STVVNPKSGSWN |
| | | | | *o*o**oooooo |
| | | | | seqid118 |
| | | | | FFDWFSGLMSWFGGPLK |
| | | | | *o***oo*o**oooooo |
| | | unclassified Phlebovirus | Anhanga virus | |
| | | (continued on next page) | Arumowot virus | |
| | | | Chagres virus | |
| | | | Corfou virus | |
| | | | Gabek Forest virus | |
| | | | Itaporanga virus | |
| | | | Phlebovirus Adria/ALB1/2005 | |
| | | | Phlebovirus Adria/ALB5/2005 | |
| | | | Phlebovirus AH12 | |
| | | | Phlebovirus AH12/China/2010 | |
| | | | Phlebovirus AH15/China/2010 | |
| | | | Phlebovirus B105-05 | |
| | | | Phlebovirus B151-04 | |
| | | | Phlebovirus B43-02 | |
| | | | Phlebovirus B68-03 | |
| | | | Phlebovirus B79-02 | |
| | | | Phlebovirus Chios-A | |
| | | | Phlebovirus Cyprus | |
| | | | Phlebovirus HB29/China/2010 | |
| | | | Phlebovirus HN13/China/2010 | |
| | | | Phlebovirus HN6/China/2010 | |
| | | | Phlebovirus Hu/Xinyangl/China/2010 | |
| | | | Phlebovirus Hu/Xinyang2/China/2010 | |
| | | | Phlebovirus IB13-04 | |
| | | | Phlebovirus JS2007-01 | |
| | | | Phlebovirus JS24 | |
| | | | Phlebovirus JS26 | |
| | | | Phlebovirus JS3/China/2010 | |
| | | | Phlebovirus JS4/China/2010 | |
| | | | Phlebovirus JS6 | |
| | | | Phlebovirus JSD1 | |
| | | | Phlebovirus LN2/China/2010 | |
| | | | Phlebovirus LN3/China/2010 | |
| | | | Phlebovirus sandflies/Gr29/Spain/2004 | |
| | | | Phlebovirus sandflies/Gr36/Spain/2004 | |
| | | | Phlebovirus sandflies/Gr44/Spain/2004 | |
| | | | Phlebovirus sandflies/Gr49/Spain/2004 | |
| | | | Phlebovirus sandflies/Gr52/Spain/2004 | |
| | | | Phlebovirus sandflies/Gr65/Spain/2004 | |
| | | | Phlebovirus sandflies/Gr98/Spain/2004 | |
| | | | Phlebovirus SD24/China/2010 | |
| | | | Phlebovirus SD4/China/2010 | |
| | | | Phlebovirus tick/XCQ-2011 | |
| | | | Phlebovirus XLL/China/2009 | |
| | | | Rio Grande virus | |
| | | | Salobo virus | |
| | | | Sandfly fever sicilian virus | |
| | | | Sandfly Sicilian Turkey virus | |
| | | | Utique virus | |
| | | | Phlebovirus sp. | |
| | | | Phlebovirus sp. Be An 24262 | |
| | | | Phlebovirus sp. Be An 356637 | |
| | | | Phlebovirus sp. Be An 416992 | |
| | | | Phlebovirus sp. Be An 578142 | |
| | | | Phlebovirus sp. Be Ar 371637 | |
| | | | Phlebovirus sp. Co Ar 170255 | |
| | | | Phlebovirus sp. Co Ar 171616 | |
| | | | Phlebovirus sp. GML 902878 | |
| | | | Phlebovirus sp. Pa Ar 2381 | |
| | | | Phlebovirus sp. PAN 479603 | |
| | | | Phlebovirus sp. PAN 483391 | |
| | | | Phlebovirus sp. VP-161A | |
| | | | Phlebovirus sp. VP-334K | |
| | | | Phlebovirus sp. VP-366G | |
| Orthomyxo- | Influenzavirus | Influenza A | INFA H1 | seqid119 |
| viridae | A virus | | | GLFGAIAGFIEGGWTG |
| | | | | seqid178 |
| | | | | WTYNAELLVLLENERTLD |
| | | | | seqid179 |
| | | | | NAELLVLLENERTLDYHD |
| | | | INFA H2 | seqid120 |
| | | | | GLFGAIAGFIEGGWQG |
| | | | | seqid180 |
| | | | | WTYNAELLVLMENERTLD |
| | | | | seqid181 |
| | | | | NAELLVLMENERTLDYHD |
| | | | INFA H3 | seqid121 |
| | | | | GIFGAIAGFIENGWEG |
| | | | | seqid182 |
| | | | | WSYNAELLVALENQHTID |
| | | | | seqid183 |
| | | | | NAELLVALENQHTIDLTD |
| | | | INFA H4 | seqid122 |
| | | | | GLFGAIAGFIENGWQG |
| | | | | seqid182 |
| | | | | WSYNAELLVALENQHTID |
| | | | | seqid184 |
| | | | | NAELLVALENQHTIDVTD |
| | | | INFA H5 | seqid120 |
| | | | | GLFGAIAGFIEGGWQG |
| | | | | seqid180 |
| | | | | WTYNAELLVLMENERTLD |
| | | | | seqid185 |
| | | | | NAELLVLMENERTLDFHD |
| | | | INFA H6 | seqid123 |
| | | | | GIFGAIAGFIEGGWTG |
| | | | | seqid119 |
| | | | | GLFGAIAGFIEGGWTG |
| | | | | seqid178 |
| | | | | WTYNAELLVLLENERTLD |
| | | | | seqid186 |
| | | | | NAELLVLLENERTLDMHD |
| | | | INFA H7 | seqid187 |
| | | | | WSYNAELLVAMENQHTID |
| | | | | seqid208 |
| | | | | WSYNAELLVAMENQHLAD |
| | | | INFA H8 | seqid124 |
| | | | | GLFGAIAGFIEGGWSG |
| | | | | seqid189 |
| | | | | WAYNAELLVLLENQKTLD |
| | | | | seqid190 |
| | | | | NAELLVLLENQKTLDEHD |
| | | | INFA H9 | seqid125 |
| | | | | GLFGAIAGFIEGGWPG |
| | | | | seqid124 |
| | | | | GLFGAIAGFIEGGWSG |
| | | | | seqid189 |
| | | | | WAYNAELLVLLENQKTLD |
| | | | | seqid190 |
| | | | | NAELLVLLENQKTLDEHD |
| | | | INFA H10 | seqid191 |
| | | | | WTYQAELLVAMENQHTID |
| | | | | seqid192 |
| | | | | QAELLVAMENQHTIDMAD |
| | | | INFA H11 | seqid125 |
| | | | | GLFGAIAGFIEGGWPG |
| | | | | seqid193 |
| | | | | WSYNAQLLVLLENEKTLD |
| | | | | seqid194 |
| | | | | NAQLLVLLENEKTLDLHD |
| | | | INFA H12 | seqid125 |
| | | | | GLFGAIAGFIEGGWPG |
| | | | | seqid189 |
| | | | | WAYNAELLVLLENQKTLD |
| | | | | seqid190 |
| | | | | NAELLVLLENQKTLDEHD |
| | | | INFA H13 | seqid125 |
| | | | | GLFGAIAGFIEGGWPG |
| | | | | seqid195 |
| | | | | WSYNAKLLVLLENDKTLD |
| | | | | seqid196 |
| | | | | NAKLLVLLENDKTLDMHD |
| | | | INFA H14 | seqid122 |
| | | | | GLFGAIAGFIENGWQG |
| | | | | seqid182 |
| | | | | WSYNAELLVALENQHTID |
| | | | | seqid184 |
| | | | | NAELLVALENQHTIDVTD |
| | | | INFA H15 | seqid187 |
| | | | | WSYNAELLVAMENQHTID |
| | | | | seqid188 |
| | | | | NAELLVAMENQHTIDLAD |
| | | | INFA H16 | seqid125 |
| | | | | GLFGAIAGFIEGGWPG |
| | | | | seqid197 |
| | | | | WSYNAKLLVLIENDRTLD |
| | | | | seqid198 |
| | | | | NAKLLVLIENDRTLDLHD |
| | Influenza- | Influenza B | All strains | seqid126 |
| | virus B | virus | | GFFGAIAGFLEGGWEG |
| | | | | seqid199 |
| | | | | ISSQIELAVLLSNEGIIN |
| | | | | seqid200 |
| | | | | QIELAVLLSNEGIINSED |
| | Influenza | Influenza C | | |
| | virus C | virus | | |
| Paramyxo- | Paramyxovirinae | Avulavirus | Avian paramyxovirus 2 Yucaipa virus | seqid127 |
| viridae | | | Avian paramyxovirus 3 | GAIALGVATAAAVTAG |
| | | | Avian paramyxovirus 3b | oooo*o*oo*o*oo** |
| | | | Avian paramyxovirus 4 | |
| | | | Avian paramyxovirus 5 | |
| | | | Avian paramyxovirus 6 | |
| | | | Avian paramyxovirus 7 | |
| | | | Avian paramyxovirus 8 | |
| | | | Avian paramyxovirus 9 | |
| | | | Newcastle disease virus | |
| | | | Pigeon paramyxovirus 1 | |
| | | | unclassified Avulavirus | |
| | | | Avian paramyxovirus 10_Avian | |
| | | | paramyxovirus duck/Miyagi/885/05 | |
| | | | Avian paramyxovirus penguin/Falkland | |
| | | | Islands/324/2007 | |
| | | | Goosramyxovirus HZ | |
| | | | Goose paramyxovirus JS/1/97/Go | |
| | | | Goose paramyxovirus SF02 | |
| | | Henipavirus | Hendra virus Hendra virus | |
| | | | horse/Australia/Hendra/1994 | |
| | | | Nipah virus | |
| | | | unclassified Henipavirus | |
| | | | Bat paramyxovirus | |
| | | | Eid.hel/GH45/2008 | |
| | | Morbillivirus | Canine distemper virus | |
| | | | Cetacean morbillivirus_Dolphin | |
| | | | morbillivirus_Pilot whale | |
| | | | morbillivirus Porpoise morbillivirus | |
| | | | Measles virus | |
| | | | Peste-des-petits-ruminants virus | |
| | | | Phocine distemper virus | |
| | | | Phocine distemper virus 1 | |
| | | | Phocine distemper virus-2 | |
| | | | Rinderpest virus | |
| | | Respirovirus | Bovine parainfluenza virus 3 | |
| | | | Porcine paramyxovirus strain Frost | |
| | | | Porcine paramyxovirus strain Texas | |
| | | | Human parainfluenza virus 1 | |
| | | | Human parainfluenza virus 3 | |
| | | | Simian Agent 10 | |
| | | | Sendai virus | |
| | | | unclassified Respirovirus | |
| | | | Atlantic salmon respirovirus | |
| | | | Guinea pig parainfluenza virus TS-9 | |
| | | | Pacific salmon paramyxovirus | |
| | | | Trask River 1983 Swine parainfluenza | |
| | | | virus 3 | |
| | | | Tursiops truncatus parainfluenza | |
| | | | virus 1 | |
| | | Rubulavirus | Human parainfluenza virus 2 | |
| | | | Human parainfluenza virus 2 (strain | |
| | | | Greer) | |
| | | | Human parainfluenza virus 2 (strain | |
| | | | Toshiba) | |
| | | | Human parainfluenza virus 4 | |
| | | | Human parainfluenza virus 4a | |
| | | | Human parainfluenza virus 4b | |
| | | | Mapuera virus | |
| | | | Mumps virus | |
| | | | Parainfluenza virus 5 | |
| | | | Porcine rubulavirus | |
| | | | Simian virus 41 | |
| | | | unclassified Rubulavirus | |
| | | | Porcine parainfluenza virus | |
| | | | Tuhoko virus 1 | |
| | | | Tuhoko virus 2 | |
| | | | Tuhoko virus 3 | |
| | | unclassified | Atlantic salmon paramyxovirus | |
| | | Paramyxovirinae | Beilong virus | |
| | | | Canine parainfluenza virus | |
| | | | Chimeric human parainfluenza virus | |
| | | | rPIV3-2 | |
| | | | Fer-de-lance virus | |
| | | | J-virus | |
| | | | Menangle virus | |
| | | | Mossman virus | |
| | | | Murayama virus | |
| | | | Ovine parainfluenza virus 3 | |
| | | | Pacific salmon paramyxovirus | |
| | | | Paramyxovirus GonoGER85 | |
| | | | Recombinant PIV3/PIV1 virus | |
| | | | Reptilian paramyxovirus | |
| | | | Salem virus | |
| | | | Salmo salar paramyxovirus | |
| | | | Snake ATCC-VR-1408 paramyxovirus | |
| | | | Snake ATCC-VR-1409 paramyxovirus | |
| | | | Tioman virus | |
| | | | Tupaia paramyxovirus | |
| | Pneumovirus | Human | Human respiratory syncytial virus A | seqid128 |
| | | respiratory | Human respiratory syncytial virus | FLGLILGLGAAVTAGVA |
| | | syncytial virus | (strain RSB1734) | ***oo**o*o*ooo*o* |
| | | | Human respiratory syncytial virus | seqid129 |
| | | | (strain RSB5857) | TNEAVVSLTNGMSVL |
| | | | Human respiratory syncytial virus | **o*****o**o*** |
| | | | (strain RSB6190) | seqid130 |
| | | | Human respiratory syncytial virus | VIRFQQLNKRLLE |
| | | | (strain RSB6256) | **o***o*o**** |
| | | | Human respiratory syncytial virus | seqid131 |
| | | | (strain RSB642) | REFSSNAGLT |
| | | | Human respiratory syncytial virus | ****o***o* |
| | | | (strain RSB6614) | seqid132 |
| | | | Human respiratory syncytial virus A | MLTDRELTSIVGGM |
| | | | strain Long LinkOut | ***oo**o*oooo* |
| | | | Human respiratory syncytial virus A2 | seqid133 |
| | | | Human respiratory syncytial virus B | YVIQLPLFGVMDTDCW |
| | | | Human respiratory syncytial | *oo***oo**o**o** |
| | | | virus (subgroup B / strain 18537) | seqid134 |
| | | | Human respiratory syncytial virus | CLARADNGWYCHNAGSLSYFP |
| | | | (subgroup B / strain 8/60) | **ooo*o**o*o****o*o** |
| | | | Human Respiratory syncytial virus | seqid135 |
| | | | 9320 | DTLKSLTVPVTSRECN |
| | | | Human respiratory syncytial virus B1 | **oo***o*ooooo** |
| | | | Human respiratory syncytial virus S2 | seqid136 |
| | | | Human respiratory syncytial virus | YDCKISTSKTYVSTAVLTTMG |
| | | | strain RSS-2 | *o*o*o***ooo*oo*o*oo* |
| | | | unclassified Human respiratory | seqid137 |
| | | | syncytial virus | VSCYGHNSCTVIN |
| | | | | *****ooo**oo* |
| | | | | Seqid75 |
| | | | | GIIRTLPDGCHYISNKGVDRVQVGNTVY |
| | | | | YLSKEVGK |
| | | | | ***o*ooo**o*o**o*o*o*o****o* |
| | | | | *oo*oo** |
| | | | | seqid139 |
| | | | | PLSFPDDKFDVAIRDVEHSINQTRTFLK |
| | | | | ASDQLL |
| | | | | **o**o*o*ooo*oo*ooo***ooo*oo |
| | | | | o**o** |
| | | | | seqid140 |
| | | | | KIMTSKTDISSSVITSIGAIVSCYG |
| | | | | o*o***ooo*oo*o*oo*oo***** |
| | | Bovine | All strains | seqid128 |
| | | respiratory | | FLGLILGLGAAVTAGVA |
| | | syncytial virus | | ***oo**o*o*ooo*o* |
| | Metapneumo- | Avian | All strains | CLARADNGWYCHNAGSLSYFP |
| | virus | metapneumo-virus | | **ooo*o**o*o****o*o** |
| | | Human | All strains | seqid133 |
| | | metapneumo-virus | | YVIQLPLFGVMDTDCW |
| | | | | *oo***oo**o**o** |
| Corona- | Coronavirinae | Alphacorona- | Alphacoronavirus 1 | seqid141 |
| viridae | | virus | Coronavirus group 1b | RSAIEDLLFDKVKLSDVG |
| | | | Human coronavirus 229E | **oo****oo**ooo*o* |
| | | | Human coronavirus NL63 | seqid142 |
| | | | Miniopterus bat coronavirus 1 | VPFYLNVQYRINGLGVT |
| | | | Miniopterus bat coronavirus HKU8 | o**ooooo**o**o*** |
| | | | Porcine epidemic diarrhea virus | seqid143 |
| | | | Rhinolophus bat coronavirus HKU2 | VLSQNQKLIANAFNNALHAIQ |
| | | | Scotophilus bat coronavirus 512 | **oo***o*ooo*oo*ooo** |
| | | | unclassified Alphacoronavirus | seqid144 |
| | | Betacorona- | Betacoronavirus 1 | TNSALVKIQAVVNANA |
| | | virus | Coronavirus group 2b | *oo**o*o*o***oo* |
| | | | Coronavirus group 2c | seqid145 |
| | | | Human coronavirus HKU1 | AEAQIDRLINGRLTALNAYVSQQL |
| | | | Murine coronavirus | *oo******o***oo*oo*oo*** |
| | | | Pipistrellus bat coronavirus HKU5 | seqid146 |
| | | | Rousettus bat coronavirus HKU9 | SAAQAMEKVNECVKSQSSRINFCGNGNH |
| | | | Severe acute respiratory syndrome- | IIS |
| | | | related coronavirus recombinant | o*oo*oo*oo***oo*oo*oo***o*o* |
| | | | SARSr-CoV | oo* |
| | | | SARS coronavirus | seqid147 |
| | | | Tylonycteris bat coronavirus HKU4 | APYGLYFIHFNYVP |
| | | | unclassified Betacoronavirus | **o*oo*o*oo*o* |
| | | Gammacorona- | Avian coronavirus | seqid148 |
| | | virus | Beluga Whale coronavirus SW1 | LQEAIKVLNHSYINLKDIGTYEYYVKWP |
| | | unclassified | Alpaca coronavirus CA08-1/2008 | WYVW |
| | | coronaviruses | Bat coronavirus | oo*oo*o**o*ooo*ooo*oo*o*o*** |
| | | | Bird droppings coronavirus | **o* |
| | | | Bovine respiratory coronavirus | seqid209 |
| | | | Chicken enteric coronavirus | EVFAQVKQMYKTPTLKYFGGFNFSQIL |
| | | | Coronavirus Anas | seqid210 |
| | | | Coronavirus | EVFAQVKQMYKTPAIKDFGGFNFSQIL |
| | | | oystercatcher/p17/2006/GBR | Seqid211 |
| | | | Coronavirus red knot/p60/2006/GBR | SFIEDLLFNKVTLADAGF |
| | | | Ferret enteric coronavirus 1202 | Seqid212 |
| | | | Ferret systemic coronavirus MSU-S | SAIEDLLFNKVRLSDVGF |
| | | | Ferret systemic coronavirus WADL | Seqid213 |
| | | | Guangxi coronaviridae | SLLEDLLFNKVKLSDVGF |
| | | | Human coronavirus NO | Seqid214 |
| | | | Human enteric coronavirus strain 4408 | SAIEDLLFSKVKLADVGF |
| | | | Kenya bat coronavirus | Seqid215 |
| | | | Mink coronavirus strain WD1133 | SAIEDLLFDKVKLSDVGF |
| | | | Parrot coronavirus AV71/99 | |
| | | | Quail coronavirus Italy/Elvia/2005 | |
| | | | Tai Forest coronavirus | |
| | | | unidentified coronavirus | |
| | | | unidentified human coronavirus | |
| Arena- | Arena-virus | LCMV-Lassa virus | Ippy virus | seqid149 |
| viridae | | (Old World) | Lassa virus | NALINDQLIMKNHLRDIMGIPYC |
| | | complex | Lujo virus | *o**o***o*o***o*o**o*** |
| | | | Lymphocytic choriomeningitis virus | seqid150 |
| | | | Mobala virus | FTWTLSDSEGKDTPGGYCLT |
| | | | Mopeia virus | oo*ooo*oo*ooo***o**o |
| | | | | seqid151 |
| | | | | KCFGNTAIAKCNQKHDEEFCDMLRLFDF |
| | | | | N |
| | | | | ***o*ooo****oo*oo****ooo*ooo |
| | | | | * |
| | | | | seqid152 |
| | | | | MLQKEYMERQGKTPLGLVDLFVFS |
| | | | | *ooo*oo**oo**oo*o*oooo*o |
| | | Tacaribe virus | Amapari virus | seqid150 |
| | | (New World) | Chapare virus | FTWTLSDSEGKDTPGGYCLT |
| | | complex | Flexal virus | oo*ooo*oo*ooo***o**o |
| | | | Guanarito virus | seqid151 |
| | | | Junin virus | KCFGNTAIAKCNQKHDEEFCDMLRLFDF |
| | | | Latino virus | N |
| | | | Machupo virus | ***o*ooo****oo*oo****ooo*ooo |
| | | | Oliveros virus | * |
| | | | Parana virus | seqid152 |
| | | | Pichinde virus | MLQKEYMERQGKTPLGLVDLFVFS |
| | | | Pirital virus | *ooo*oo**oo**oo*o*oooo*o |
| | | | Sabia virus | |
| | | | Tacaribe virus | |
| | | | Tamiami virus | |
| | | | Whitewater Arroyo virus | |
| Hepadna- | Genus | Hepatitis B | HBV genotype A | seqid153 |
| viridae | Orthohepadnavirus | virus | HBV genotype B | FNPLGFFPSHQLDPLF |
| | | | HBV genotype C | o***o*o*o*o*o*o* |
| | | | HBV genotype D | seqid154 |
| | | | HBV genotype E | ADWDKNPNKDPWP |
| | | | HBV genotype F | o*o*o*oo*oooo |
| | | | HBV genotype G | seqid155 |
| | | | HBV genotype H | MESITSGFLGPLLVLQAVFF |
| | | | Hepatitis B virus alpha1 | oooooooo*ooooo**oooo |
| | | | Hepatitis B virus LSH/chimpanzee | seqid156 |
| | | | Hepatitis B virus strain cpz | LLTRILTIPQSLDSWWTSLNFLGGA |
| | | | Hepatitis B virus subtype adr | oooooo*oooo*oooo***o*o*oo |
| | | | Hepatitis B virus subtype adw | seqid157 |
| | | | Hepatitis B virus subtype adyw | CPPTCPGYRWMC |
| | | | Hepatitis B virus subtype ayw | oo*o*****o*o |
| | | | | seqid158 |
| | | | | LFILLLCLIFLLVLLDYQ |
| | | | | *oo*ooo*oo*oo*oooo |
| Rhabdo- | Dimarhabdovirus | Ephemerovirus | Bovine ephemeral fever virus | seqid160 |
| viridae | | | | LDGYLCRKQKWEVTCTETWYFVTD |
| | | | | *o*oo****o*ooo*o*****o*o |
| | | | | seqid161 |
| | | | | KYQIIEVIPTENEC |
| | | | | o***o**o*oooo* |
| | | | | seqid162 |
| | | | | LKGEYIPPYYPPTNCVWNAIDTQE |
| | | | | oo*oo*******oo*o**oooo** |
| | | | | seqid163 |
| | | | | IEDPVTMTLMDSKFTKPC |
| | | | | ooo*oooooo**o*oo** |
| | | | | seqid164 |
| | | | | LHCQIKSWECIPV |
| | | | | o**oo*o****o* |
| | | | | seqid165 |
| | | | | SHRNMMEALYLESPD |
| | | | | *oo*oo*o*oo*o** |
| | | | | seqid166 |
| | | | | LTFCGYNGILLDNGEWWSIY |
| | | | | o****oo**oooo****** |
| | | | | seqid167 |
| | | | | ELEHEKCLGTLEKLQNGE |
| | | | | *****o**o*oo*oo*o* |
| | | | | seqid168 |
| | | | | LDLSYLSPSNPGKHYAY |
| | | | | **o***o*oo**oo*** |
| | | | | seqid169 |
| | | | | IRAVCYYHTFSMNLD |
| | | | | o**o*o*oo*oooo* |
| | | Vesiculovirus | Carajas virus | seqid170 |
| | | | Chandipura virus | EWKTTCDYRWYGPQYITHSI |
| | | | Cocal virus | o*o****o*****o*o*o* |
| | | | Isfahan virus | seqid171 |
| | | | Maraba virus | LGFPPQSCGWASVTT |
| | | | Piry virus | o****oo**oooooo |
| | | | recombinant Vesiculovirus | seqid1 |
| | | | Spring viraemia of carp virus | VQVTPHHVLVDEYTGEWVDSQFINGKC |
| | | | Vesicular stomatitis Alagoas virus | ooooo*o*oooo*o*o*o*oooooooo |
| | | | Vesicular stomatitis Indiana virus | |
| | | | Vesicular stomatitis New Jersey virus | |
| | Lyssavirus | Aravan virus | | |
| | | Australian bat | | |
| | | lyssavirus | | |
| | | Duvenhage virus | | |
| | | European bat | | |
| | | lyssavirus 1 | | |
| | | European bat | | |
| | | lyssavirus 2 | | |
| | | Irkut virus | | |
| | | Khujand virus | | |
| | | Lagos bat virus | | |
| | | Mokola virus | | |
| | | West Caucasian | | |
| | | bat virus | | |
| | | Rabies virus | Rabies virus AB21 | seqid5 |
| | | | Rabies virus AB22 | GFTCTGVVTEAETYTNFVGYVT |
| | | | Rabies virus AVO1 | *o****o**o*oo*oooo*** |
| | | | Rabies virus BNG4 | seqid6 |
| | | | Rabies virus BNG5 | SLHNPYPDYRWLRTVKTT |
| | | | Rabies virus China/DRV | *ooooooooooo***o* |
| | | | Rabies virus China/MRV | Seqid138 |
| | | | Rabies virus CVS-11 | ESLVIISPSVADLDPYDRSLHS |
| | | | Rabies virus ERA | *ooo***oooo*o**ooo |
| | | | Rabies virus Eth2003 | Seqid91 |
| | | | Rabies virus HEP-FLURY | CKLKLCGVLGLRLMDGT |
| | | | Rabies virus India | *ooo****oooo*ooo* |
| | | | Rabies virus Nishigahara RCEH | Seqid206 |
| | | | Rabies virus Ontario fox | ILGPDGNVLIPEMQSS |
| | | | Rabies virus Ontario skunk | o**o*ooo*******o |
| | | | Rabies virus PM | seqid82 |
| | | | Rabies virus red fox/08RS- | QHMELLESSVIPLVHPL |
| | | | 1981/Udine/2008 | *ooo**o*ooo**oo** |
| | | | Rabies virus SAD B19 | |
| | | | Rabies virus silver-haired bat- | |
| | | | associated SHBRV | |
| | | | Rabies virus strain Pasteur vaccin | |
| | | | Rabies virus strain Street | |
| | | | Rabies virus vnukovo-32 | |
| | | | Thailand genotype 1 dog lyssavirus | |
| | | unclassified | Bokeloh bat lyssavirus | |
| | | Lyssavirus | European bat lyssavirus | |
| | | | Lyssavirus Ozernoe | |
| | | | Shimoni bat virus | |
| | Novirhabdovirus | Hirame | | |
| | | rhabdovirus | | |
| | | Infectious | | |
| | | hematopoietic | | |
| | | necrosis virus | | |
| | | Snakehead | | |
| | | rhabdovirus | | |
| | | Viral | | |
| | | hemorrhagic | | |
| | | septicemia virus | | |
| | unassigned | Bangoran virus | | |
| | Rhabdoviridae | Bimbo virus | | |
| | | Bivens Arm virus | | |
| | | Flanders virus | | |
| | | Garba virus | | |
| | | Klamath virus | | |
| | | Malpais Spring | | |
| | | virus | | |
| | | Nasoule virus | | |
| | | Ngaingan virus | | |
| | | Ouango virus | | |
| | | Sigma virus | | |
| | | Tupaia virus | | |
| | | Wongabel virus | | |
| Filoviridae | | | Lloviu virus (LLOV) | Seqid216 |
| | | | Bundibugyo virus (BDBV; previously BEBOV) | GAAIGLAWIPYFGPAAE |
| | | | Reston virus (RESTV; previously REBOV) | oo*o*oo***o***ooo |
| | | | Sudan virus (SUDV; previously SEBOV) | seqid217 |
| | | | Tai Forest virus (TAFV; previously CIEBOV) | GAAVGLAWIPYFGPAAE |
| | | | Ebola virus (EBOV; previously ZEBOV) | Seqid218 |
| | | | Marburg virus (MARV) | GAAAGLAWIPYFGPAAE |
| | | | Ravn virus (RAW) | Seqid219 |
| | | | | DLAAGLSWIPFFGPGIE |
| | | | | Seqid220 |
| | | | | HNAAGIAWIPYFGPGAE |
| Lentivirisae | | Hiv1 | | Seqid221 |
| | | | | AVGLGALFLGFLGAAGSTMGAAS |
| | | | | oooo**ooo*o*oo*****o**o |
| | | | | seqid222 |
| | | | | LTLTGQARQLLS |
| | | | | o***o*o*o*oo |
| | | | | seqid223 |
| | | | | GIVQQQSNLLQAIEAQQ |
| | | | | o*****o***o*****o |
| | | | | seqid224 |
| | | | | GLGAMFLGFLGAAGSTMGAASLTLTVQA |
| | | | | RQLLS |
| | | | | Seqid225 |
| | | | | GIGAMFLGLLSAAGSTMSAAAITLTVQT |
| | | | | RQLLS |
| | | | | Seqid226 |
| | | | | GIGAMFLGLLSAAGSTMGAAAITLTVQT |
| | | | | RQLLS |
| | | | | Seqid227 |
| | | | | GIGAVFLGFLGAAGSTMGAASITLTVQA |
| | | | | RQLLS |
| | | | | Seqid228 |
| | | | | GVGALFLGFLSAAGSTMGAASITLTVQA |
| | | | | RQLLS |
| | | | | Seqid229 |
| | | | | GIGAMILGFLGAAGSTMGAASITLTVQA |
| | | | | RQLLS |
| | | | | Seqid230 |
| | | | | GLGAMFLGFLGAAGSTMGAASITLTVQA |
| | | | | RQLLS |
| | | | | Seqid231 |
| | | | | GFGAMFLGFLGAAGSTMGAASITLTVQA |
| | | | | RQLLS |
| | | | | Seqid232 |
| | | | | TLGAMFLGFLGAAGSTMGAASMTLTVQA |
| | | | | RQLLS |
| | | | | Seqid233 |
| | | | | GLGAVFLGFLGAAGSTMGAASITLTVQA |
| | | | | RQLLS |
| | | | | Seqid234 |
| | | | | TLGAMFLGFLGAAGSTMGAASMTLTVQA |
| | | | | RRLLS |
| | | | | Seqid235 |
| | | | | TIGAMFLGFLGAAGSTMGAASMTLTVQA |
| | | | | RLLLS |
| | | | | Seqid236 |
| | | | | TLGAMFLGFLGAAGSTMGAASMTLTVQA |
| | | | | RLLFS |
| | | | | Seqid237 |
| | | | | TLGAMFLGFLGAAGSTMGAASLTLTVQA |
| | | | | RLLLS |
| | | | | Seqid238 |
| | | | | GVGAMFLGFLGAAGSTMGAASLTLTVQA |
| | | | | RQLLS |
| | | | | Seqid239 |
| | | | | GLGAMFLGFLGAAGSTMGAASITLTVQA |
| | | | | RLLLS |
| | | | | Seqid240 |
| | | | | TLGAVFLGFLGAAGSTMGAASLTLTVQA |
| | | | | RLLLS |
| | | | | Seqid241 |
| | | | | GIGAVFLGFLGAAGSTMGAASITLTVQA |
| | | | | RKLLS |
| | | | | Seqid242 |
| | | | | GIGALFLGFLGAAGSTMGAASVTLTVQA |
| | | | | RQLLS |
| | | | | Seqid243 |
| | | | | GLGALFLGFLGAAGSTMGAASVTLTVQA |
| | | | | RQLLS |
| | | | | Seqid244 |
| | | | | GIGAMFLGFLGAAGSTMGAASITLTVQA |
| | | | | RLLLS |
| | | | | Seqid245 |
| | | | | GIGAMFLGFLGAAGSTMGAASVTLTVQA |
| | | | | RLLLS |
| | | | | Seqid246 |
| | | | | AIGALFLGFLGAAGSTMGAASVTLTVQA |
| | | | | RLLLS |
| | | | | Seqid247 |
| | | | | TLGAMFLGFLGAAGSTMGAASLTLTVQA |
| | | | | RQLLS |
| | | | | Seqid248 |
| | | | | GIGALFLGFLGAAGSTMGAASMTLTVQA |
| | | | | RQLLS |
| | | | | Seqid249 |
| | | | | GIGAMFLGFLGAAGSTMGAASLTLTVQA |
| | | | | RQLLS |
| | | | | Seqid250 |
| | | | | GIGAVFLGFLGAAGSTMGAASMTLTVQA |
| | | | | RLLLS |
| | | | | Seqid251 |
| | | | | GIGALFLGFLGAAGSTMGAASLTLTVQA |
| | | | | RQLLS |
| | | | | Seqid252 |
| | | | | GIGAVFLGILGAAGSTMGAASITLTVQA |
| | | | | RQLLS |
| | | | | Seqid253 |
| | | | | GIGAVFLGFLGAAGSTMGAASVTLTVQA |
| | | | | RQLLF |
| | | | | Seqid254 |
| | | | | GLGAMFFGFLGAAGSTMGAASVTLTVQA |
| | | | | RQLLS |
| | | | | Seqid255 |
| | | | | GIGALFLGFLGAAGSTMGAASITLTVQA |
| | | | | RLLLS |
| | | | | Seqid256 |
| | | | | GLGALFVGFLGAAGSTMGAASITLTVQA |
| | | | | RQLLS |
| | | | | Seqid257 |
| | | | | GIGALFLGFLGTAGSTMGAASVTLTVQA |
| | | | | RQLLS |
| | | | | Seqid258 |
| | | | | GIGAMIFGFLGAAGSTMGAASITLTVQA |
| | | | | RQLLS |
| | | | | Seqid259 |
| | | | | GLGAVLLGFLGTAGSTMGAASLTLTVQV |
| | | | | RQLLS |
| | | | | Seqid260 |
| | | | | GIGAVLFGFLGAAGSTMGAASITLTVQV |
| | | | | RQLLS |
| | | | | Seqid261 |
| | | | | GLGALFLGFLGAAGSTMGAASLTLTGQA |
| | | | | RQLLS |
| | | | | oo**ooo*o*oo*****o**oo***o*o |
| | | | | *o*oo |
| | | | | Seqid262 |
| | | | | GTLGAMFLGFLGAAGSTMGAASMTLTVQ |
| | | | | ARQLL |
| | | | | Seqid263 |
| | | | | GTIGAMFLGFLGAAGSTMGAASITLTVQ |
| | | | | ARRLL |
| | | | | Seqid264 |
| | | | | GTIGAMFLGFLGAAGSTMGAASMTLTVQ |
| | | | | ARLLL |
| | | | | Seqid265 |
| | | | | IGALFLGFLGAAGSTMGAASVTLTVQAR |
| | | | | LLLSG |
| | | | Bovine lentivirus group | Seqid266 |
| | | | | AVGMVIFLLVLAIMAMTASVTAA |
| | | | | ***oo**********o*o*oo** |
| | | | Equine lentivirus group | Seqid267 |
| | | | | FGISAIVAAIVAATAIAASA |
| | | | | **o*ooo**********o*o |
| | | | Feline lentivirus group | Seqid268 |
| | | | | TLALVTATTAGLIGTTTGTSA |
| | | | | Seqid269 |
| | | | | HVMLALATVLSMAGAGTGATA |
| | | | Ovine/caprine lentivirus group | Seqid270 |
| | | | | GIGLVIMLVTMAIVAAAGAS |
| | | | | *o***oo*oo***o*o***o |
| | | | Human immunodeficiency virus 2 | Seqid271 |
| | | | | GVMVLGFLGFLAMAGSAMGA |
| | | | | ooo***o**oooo*oooooo |
| | | | Simian immunodeficiency virus | Seqid272 |
| | | | | GVFVLGFLGFLATAGSAMGA |
| | | | | oooo**oo*o*oo**ooooo |
| | | | Simian immunodeficiency virus others | Seqid273 |
| | | | | GAIVLGLLGFLGLAGSAMG |
| | | | | *ooooooo*o*ooo**ooo |
| | | | Ovine lentivirus | Seqid274 |
| | | | | GIGLVIVLAIMAIIAAAGAGLGVANAVQ |
| | | Peptides from domains from | | |
| | | fusion proteins exhibiting | | |
| | | immunosuppressive activity | Name of envelope attachment/ | IU group and |
| Family | Genus | (ISU) | fusion protein | fusion type |
| Flavi- | Flavi- | seqid2 | Envelope protein prME | Group 1 Type II |
| viridae | virus | DRGWGNGCGLFGKG | Fusion protein E | Fusion mechanism |
| | | ************** | | |
| | | seqid172 | | |
| | | KGSSIGKMFESTYRGAKRMAILG | | |
| | | seqid2 | | |
| | | DRGWGNGCGLFGKG | | |
| | | ************** | | |
| | | seqid2 | | |
| | | DRGWGNGCGLFGKG | | |
| | | ************** | | |
| | | seqid204 | | |
| | | GDTAWDFGSVGGVLNSLGK | | |
| | | ******************* | | |
| | | seqid2 | | |
| | | DRGWGNGCGLFGKG | | |
| | | ************** | | |
| | | seqid2 | | |
| | | DRGWGNGCGLFGKG | | |
| | | ************** | | |
| | | seqid2 | | |
| | | DRGWGNGCGLFGKG | | |
| | | ************** | | |
| | | seqid2 | | |
| | | DRGWGNGCGLFGKG | | |
| | | ************** | | |
| | | seqid2 | | |
| | | DRGWGNGCGLFGKG | | |
| | | ************** | | |
| | | seqid2 | | |
| | | DRGWGNGCGLFGKG | | |
| | | ************** | | |
| | | seqid2 | | |
| | | DRGWGNGCGLFGKG | | |
| | | ************** | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | | seqid2 | | |
| | | DRGWGNGCGLFGKG | | |
| | Hepaci- | seqid3 | E1/E2 | |
| | virus | GLIHLHQNIVDVQYLYG | | |
| | | seqid175 | | |
| | | PALSTGLIHLHQNIVDVQ | | |
| | | seqid3 | | |
| | | GLIHLHQNIVDVQYLYG | | |
| | | seqid3 | | |
| | | GLIHLHQNIVDVQYLYG | | |
| | | seqid3 | | |
| | | GLIHLHQNIVDVQYLYG | | |
| | | seqid3 | | |
| | | GLIHLHQNIVDVQYLYG | | |
| | Pesti virus | | E1/E2 | |
| | Unnclassified | | | |
| | Flaviviridae | | | |
| Togaviridae | Alpha-virus | | | E2/E1 |
| | Rubivirus | | | |
| Bunya- | Hanta-virus | | | Gn(G2)/Gc(G1) |
| viridae | (continued on | | | |
| | next page) | | | |
| | Ortho-bunya- | | | |
| | virus | | | |
| | Phlebovirus | | | |
| Orthomyxo- | Influenzavirus | seqid4 | INF F#2 DELTA6: | HA |
| viridae | A virus | GLFGAIAGFIENGWEG | seqid201 | (HA1/HA2) |
| | | seqid4 | GLFGAAGFIENGWEG | |
| | | GLFGAIAGFIENGWEG | InFAH1-3: seqid203 | |
| | | seqid4 | | |
| | | GLFGAIAGFIENGWEG | | |
| | | seqid4 | | |
| | | GLFGAIAGFIENGWEG | | |
| | Influenza- | | | |
| | virus B | | | |
| | Influenza | | | |
| | virus C | | | |
| Paramyxo- | Paramyxovirinae | | | F0 |
| viridae | Pneumovirus | | | (F2/F1) |
| | Metapneumo- | | | |
| | virus | | | |
| Corona- | Coronavirinae | | | S |
| viridae | | | | (S1/S2) |
| Arena- | Arena-virus | | | GpC |
| viridae | | | | (Gp1/Gp2) |
| Hepadna- | Genus | | | L and M and S |
| viridae | Orthohepadnavirus | | | Where S mediates fusion |
| Rhabdo- | Dimarhabdovirus | | | Glycoprotein G |
| viridae | Lyssavirus | | | |
| | Novirhabdovirus | | | |
| | unassigned | | | |
| | Rhabdoviridae | | | |
| Filoviridae | | | | |
| Lentivirisae | | | | |

### EXAMPLES

### Peptide Solutions

The peptides were either dissolved in water or in cases of low water solubility, 5% DMSO solutions were used to dissolve the peptides.

Assay to Measure the Immunosuppressive Activity of Peptides Derived from Viral Surface Proteins or their Mutants

The peptides can be prepared by different means including, but not limited to, solid phase synthesis commonly used for such purposes. The peptides can be dimerized using a cysteine residue either at the N- or C-terminal or in the middle of the peptide or by using any other molecule or atom that is covalently bound to peptide molecules.

The peptides can be coupled to a carrier protein such as BSA by covalent bounds including, but not limited to, disulfide bridges between the peptide cysteine residues and the carrier protein or through amino groups including those in the side chain or Lysine residues.

The peptides can have non-viral derived amino acids added to their C-terminal for increasing their water solubility.

### Assay to Test the Immunosuppressive Activity of Peptides

### Experiment Design

Human Peripheral Blood Mononuclear Cells (PBMC) are prepared freshly from healthy donors. These are stimulated by Con A (5 ug/mL) concomitant to peptide addition at different concentrations (i.e. 25 uM, 50 uM and 100 uM). Cultures are maintained and lymphocyte proliferation is measured 72 hrs later by EdU incorporation and Click-iT labelling with Oregon Green (Invitrogen, Denmark) as recommended by the manufacturer. The degree of activated lymphocytes is proportional to the fluorescence detection.

### CTLL-2 Assay

100.000 CTLL-2 cells are seeded pr. well in a 48 well-plate (Nunc) in 200 uL of medium (RPMI+2 mM L-glutamine+1 mM Na-pyruvat+10% FCS+0.5 ng/mL IL-2) 2 hours later the peptides are added to the wells. 24 h later the cells are labeled using the Click-it reaction kit (Invitrogen cat. # C35002). The fluorescence of the cells is measured on a flow cytometer. The degree of proliferation in each sample is proportional to the detected fluorescence.

Test of Immunosuppression from Monomer and Dimeric Peptides

100.000 CTLL-2 cells were seeded pr. well in a 48 well-plate (Nunc) in 200 uL of medium (RPMI+2 mM L-glutamine+1 mM Na-pyruvat+10% FCS+0.5 ng/mL IL-2) 2 hours later the peptides were added to the wells. 24 h later the cells were labeled using the Click-it reaction kit (Invitrogen cat. # C35002). The fluorescence of the cells was measured on a flow cytometer. The degree of proliferation in each sample is proportional to the detected fluorescence.

### Quantification of Proliferation Inhibition

The degree of inhibition of proliferation of CTLL-2 cells is visualized in the diagrams in the figures. The ratios are calculated by dividing the number of labeled cells (growing cells) in cultures in presence of peptide with cultures in absence of peptides, but added the same volume of the solute that was used to dissolve the peptides. That is in cases where the peptides were dissolved in 5% DMSO, the same volume of 5% DMSO was added to the control cells.

### FIGURES

FIG. 1 shows the result of an experiment using Influenza derived peptide. The dimeric peptide inhibits the proliferation of CTLL-2 cells, where as the monomer even at higher concentration has no effect. Interestingly the mixing of the monomer with the dimeric peptides completely removes the suppressive activity of the dimers, showing that the monomeric peptide function as an inhibitor of the suppression activity.

The peptide used has the following sequence:

IN F#2:
[Seq id 275]
GLFGAIAGFIENGWEGCGGEKEKEK

FIG. 2 shows the result of two independent experiments on Flavi virus derived peptides.

FLV IS/1 and FLV IS/2 are two independent experiments using the dimerized peptide: In both cases, a significant inhibition of proliferation of CTLL-2 cells is evident, while the monomeric peptide has no effect.

FLV IS/1 and FLV IS/2:
dimeric
[seq id 2]
DRGWGNGCGLFGKG
FLV IS mono/1:
monomeric
[seq id 2]
DRGWGNGCGLFGKG

Control peptide: a dimerized non-immune suppressive control peptide.

The concentrations are given in μM.

FIG. 3 shows that while the dimeric peptides (through ss bond at the C-terminal Cys residues) inhibit proliferation f the CTLL-2 cells, the monomeric peptides show no effect. Ebo Z monomer was not tested at 50 uM. The Dimers showed complete inhibition.

Ebo R:
[Seq id 276]
LLNRKAIDFLLQRWGGTC
Ebo Z:
[Seq id 277]
ILNRKAIDFLLQRWGGTC
Ebo W14R:
[Seq id 278]
ILNRKAIDFLLQRRGGTC

FIG. 4 shows inflammation-related enzyme and transcription factor gene expression kinetics of THP-1 monocytes stimulated with 1 μg/ml LPS. Gene expression was expressed as relative gene expression towards RPL13a-expression and non-stimulated cells at time zero (ΔΔCt). Data shown are means+standard deviation from two independent biological replications.

FIG. 5 shows effects of influenza dimeric ISD peptide (IN F#2; seq id 275) on expression of NF-kappaB mRNA in LPS-stimulated THP-1 cells. THP-1 cells were incubated with either medium alone, 30 μM, 60 μM INF ISD peptide or 30 μM, 60 μM control peptide, and stimulated with 1 μg/ml LPS. Data shown are the medians±standard deviation from two independent biological replications.

FIG. 6 shows effects of influenza dimeric ISD peptide (IN F#2; seq id 275) on expression of SP-1 mRNA in LPS-stimulated THP-1 cells. THP-1 cells were incubated with either medium alone, 30 μM, 60 μM INF ISD peptide or 30 μM, 60 μM control peptide, and stimulated with 1 μg/ml LPS. Data shown are the medians±standard deviation from two independent biological replications.

FIG. 7 shows effects of dimeric ISD peptide (IN F#2; seq id 275) on protein secretion of IL-8 in LPS-stimulated THP-1 cells. THP-1 cells were incubated with either medium alone, 30 μM or 60 μM INF ISD peptide or 30 μM, 60 μM control peptide, and stimulated with 1 μg/ml LPS. Data shown are the median±standard deviation from three independent experiments performed in duplicates.

FIG. 8 shows effects of dimeric ISD peptide (IN F#2; seq id 275) on protein secretion of IL-10 in LPS-stimulated THP-1 cells. THP-1 cells were incubated with either medium alone, 30 μM or 60 μM INF ISD peptide or 30 μM, 60 μM control peptide, and stimulated with 1 μg/ml LPS. Data shown are the median±standard deviation from three independent experiments performed in duplicates.

FIG. 9 shows effect of different stimulus on the secretion of IFN-gamma in PBMCs. PBMCs were incubated either with 1 μg/ml or 50 ng/ml PMA and 1 μg/ml ionomycin or 10 ng/ml SEB for indicated time periods. Data shown are the medians±standard deviation from three independent technical replicates.

FIG. 10 shows expression kinetics of IFN gamma expression in response to PMA/ionomycin treatment. Gene expression was expressed as relative gene expression towards RPL13a expression and non-stimulated cells at time zero (ΔΔCt). Data shown are the medians±standard deviation from three independent technical replicates.

FIG. 11 shows effect of dimeric ISD peptide (IN F#2; seq id 275) on secretion of protein of IFN-gamma in PMA/ionomycin stimulated PBMCs. PBMCs were incubated with either medium alone, 30 μM or 60 μM Flu ISU or 30 μM or 60 μM control peptide, and stimulated with 50 ng/ml PMA and 1 μg/ml ionomycin. Data shown are the medians±standard deviation from three independent experiments performed in duplicates.

FIG. 12 shows effects of SARS ([Seq id 279] AEVQIDRLITGRLQSLQTYVCGGEKEKEK) or Filo ISD ([Seq id 280] GAAIGLAWIPYFGPAAECGGEKEKEK) on expression of TNF-alpha mRNA in LPS-stimulated THP-1 cells. THP-1 cells were incubated with either medium alone, 30 μM, 60 μM SARS or Filo ISD peptide or 30 μM, 60 μM control peptide, and stimulated with 1 μg/ml LPS. Data shown are the medians±standard deviation from two independent biological replications.

FIG. 13 shows effects of dimeric SARS ([Seq id 279] AEVQIDRLITGRLQSLQTYVCGGEKEKEK) or Filo ISD ([Seq id 280] GAAIGLAWIPYFGPAAECGGEKEKEK) on expression of IL-1 β mRNA in LPS-stimulated THP-1 cells. THP-1 cells were incubated with either medium alone, 30 μM, 60 μM SARS or Filo ISD peptide or 30 μM, 60 μM control peptide, and stimulated with 1 μg/ml LPS. Data shown are the medians±standard deviation from two independent biological replications.

FIG. 14 shows effects of dimeric SARS or Filo ISD on expression of IL-1 β mRNA in LPS-stimulated THP-1 cells. THP-1 cells were incubated with either medium alone, 30 μM, 60 μM SARS or Filo ISD peptide or 30 μM, 60 μM control peptide, and stimulated with 1 μg/ml LPS. Data shown are the medians±standard deviation from two independent biological replications.

FIG. 15 shows interactions between dimeric ISD peptide (IN F#2; seq id 275) and STING depends on distinct STING domains. To investigate further the interaction between STING and dimeric ISD peptide (IN F#2; seq id 275) the C-terminal domian of STING was expressed with a HA-tag in HEK293 cells. STING was either in a wt form or with deletions. Lysates from tansfected cells were used for pulldown using biotinylated dimeric ISD peptide (IN F#2; seq id 275) and streptavidin coated beads. The bead eluate was then immunoblotted using antibodies against HA-tag. As seen in the figure wt STING and the deletion mutant DN5 (162-N) was readily pulled down using dimeric ISD peptide (IN F#2; seq id 275) whereas the deletion mutants DN6 (172-N) was not. These data indicate that amino acids 162-172 are necessary for interactions between dimeric ISD peptide (IN F#2; seq id 275) and STING.

FIGS. 16 and 17 show the serum IgG levels as well as IFN-γ secreting CD8+ T cell counts in animals vaccinated with influenza VLPs alone or influenza VLPs together with monomeric INF F#2 C17G ([Seq id 281] GLFGAIAGFIENGWEGGGGEKEKEK) peptide adjuvant (a control group receiving only PBS was also included) according to the study design below. Each group contained 9 animals.

| Study | |
|---|---|
| Day | Activity |
| D 1 | 1. Immunization |
| | wt VLP 2.1 μg (300 μl) s.c. |
| | wt VLP 2.1 μg + 20 μg INF F#2 C17G (300 μl) s.c. |
| | Vehicle |
| D 21 | Collection of blood and serum preparation |
| D 22 | 2. Immunization |
| | wt VLP 2.1 μg (300 μl) s.c. |
| | wt VLP 2.1 μg + 20 μg INF F#2 C17G (300 μl) s.c. |
| | INF F#2 C17G has the sequence: |
| | [Seq id 281] GLFGAIAGFIENGWEGGGGEKEKEK |
| | Vehicle |
| D 42 | Collection of blood and serum preparation |
| | Collection of spleens for ELISPOT from 3 mice/group |

FIG. 16 Serum IgG1 and IgG2a ELISA

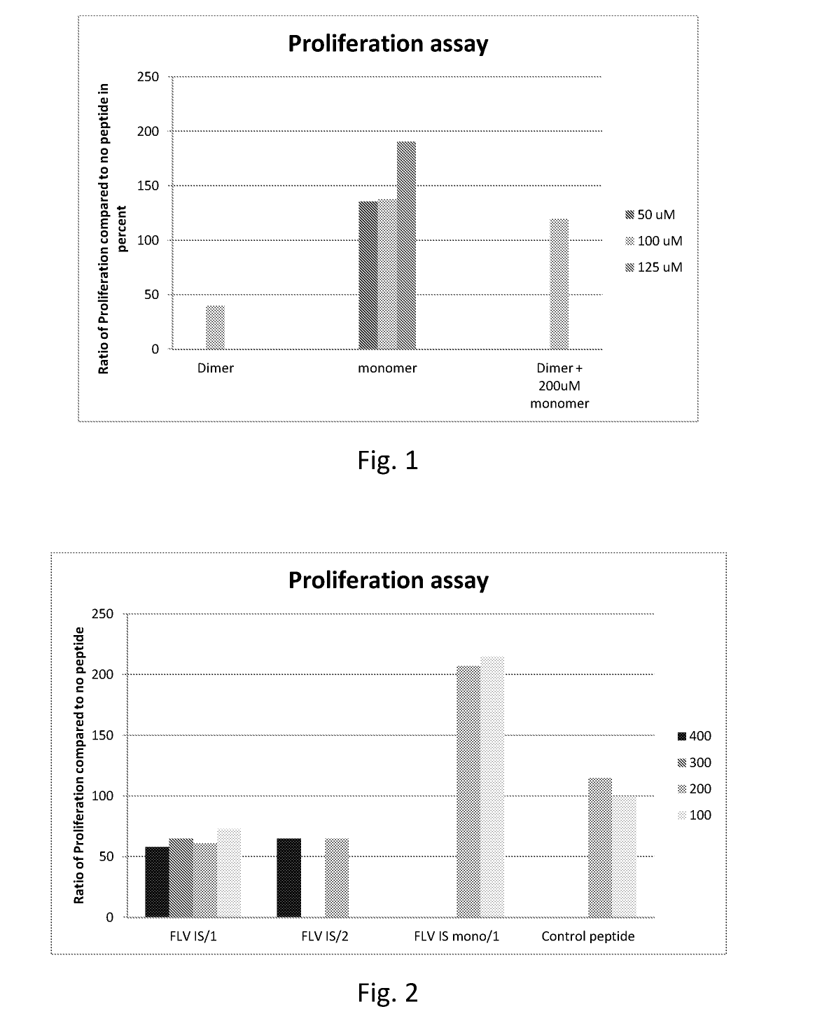

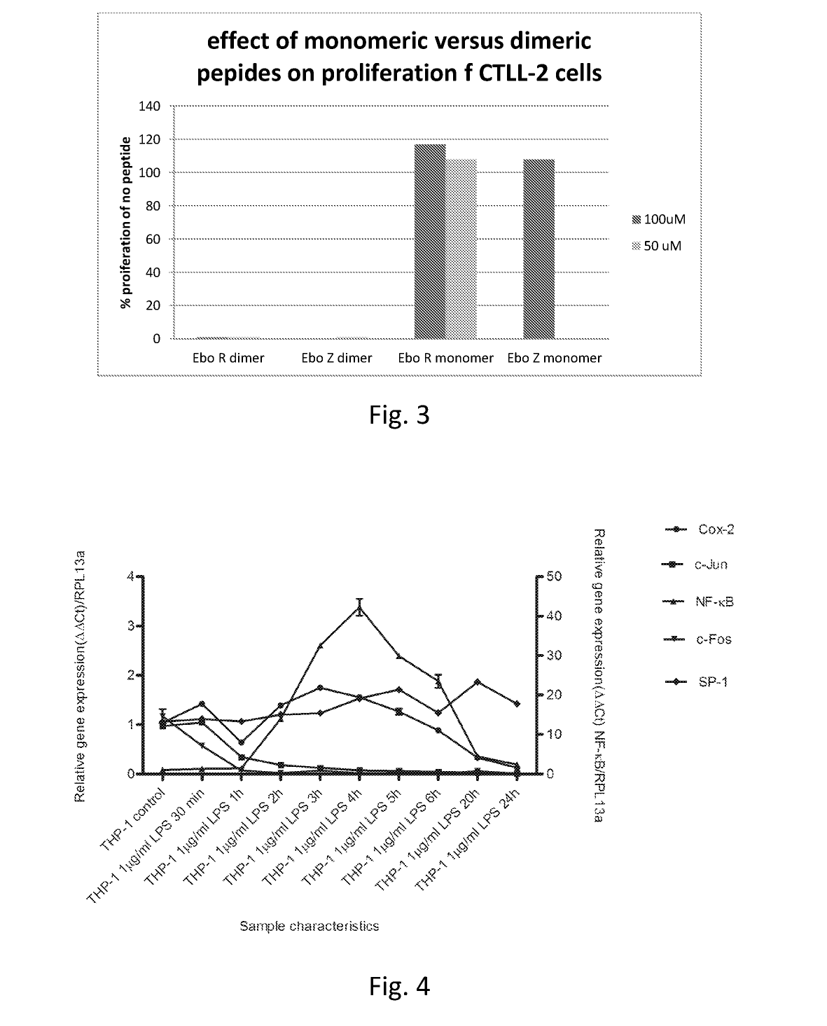

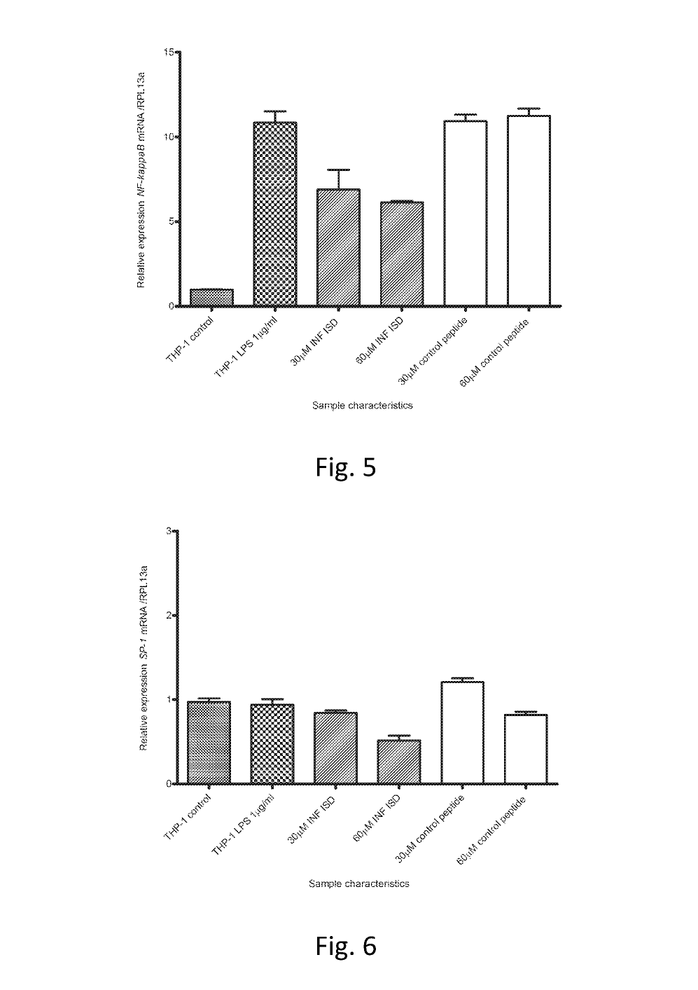

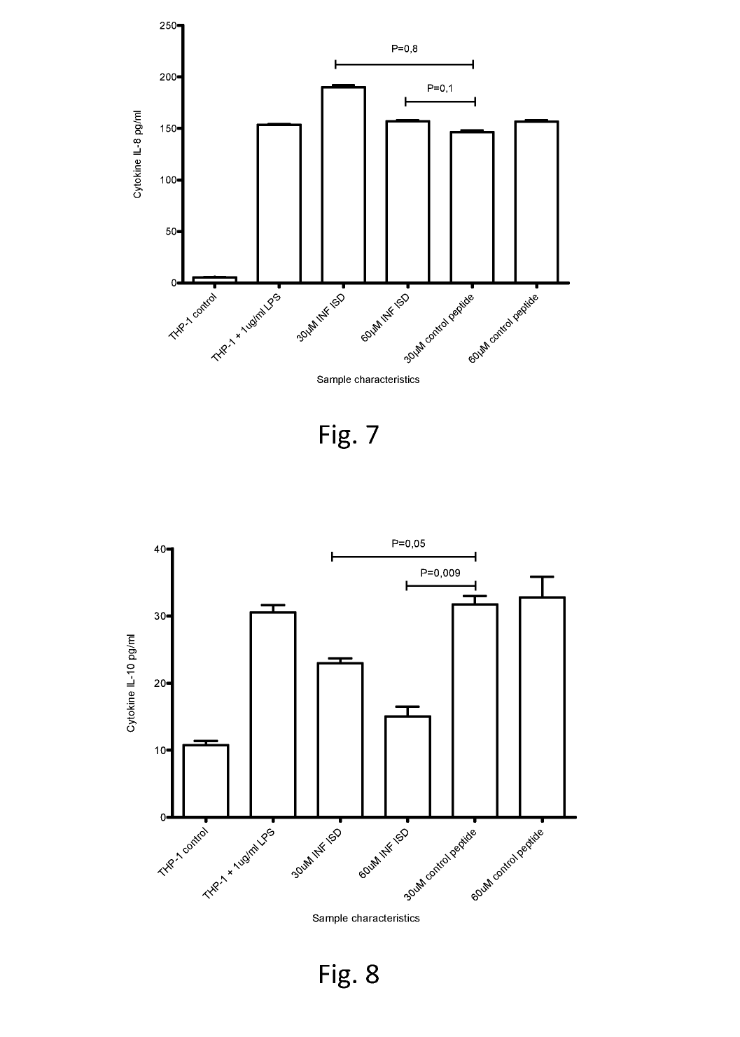

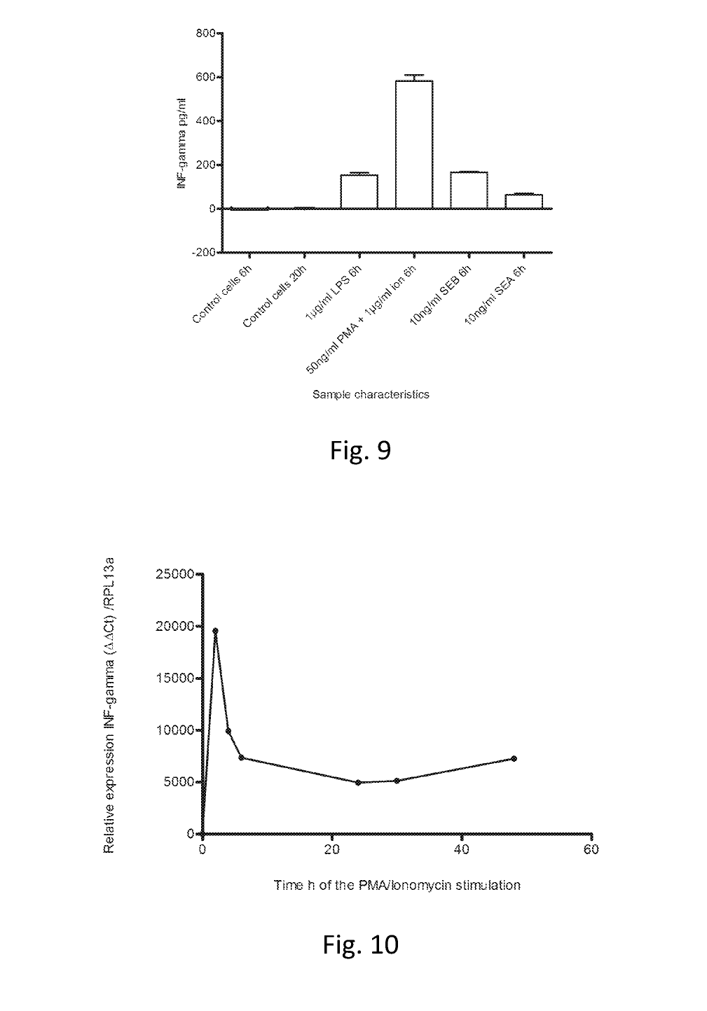

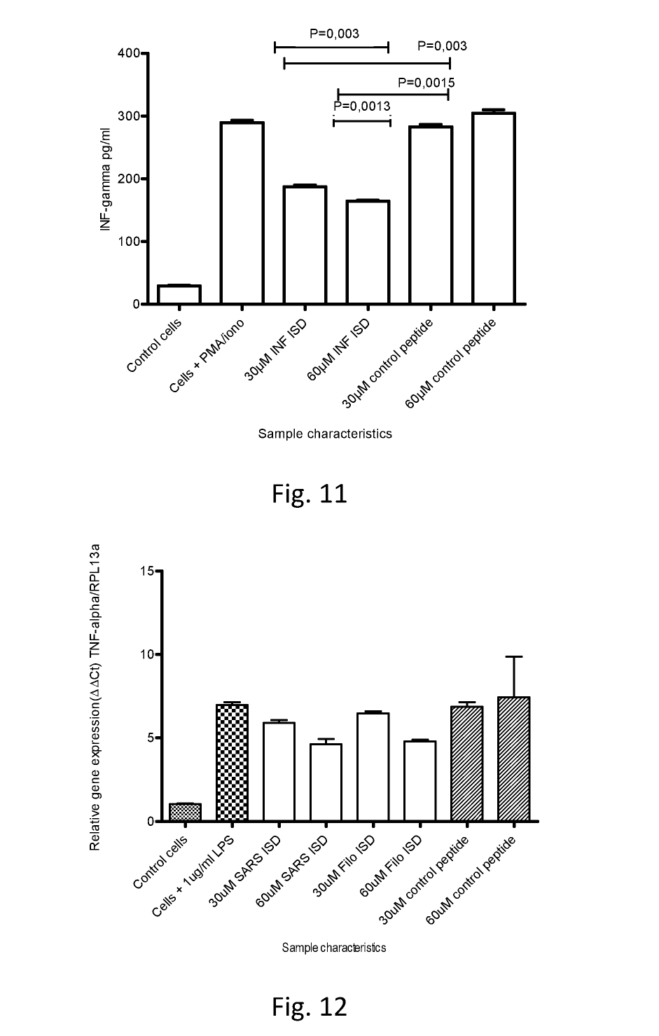

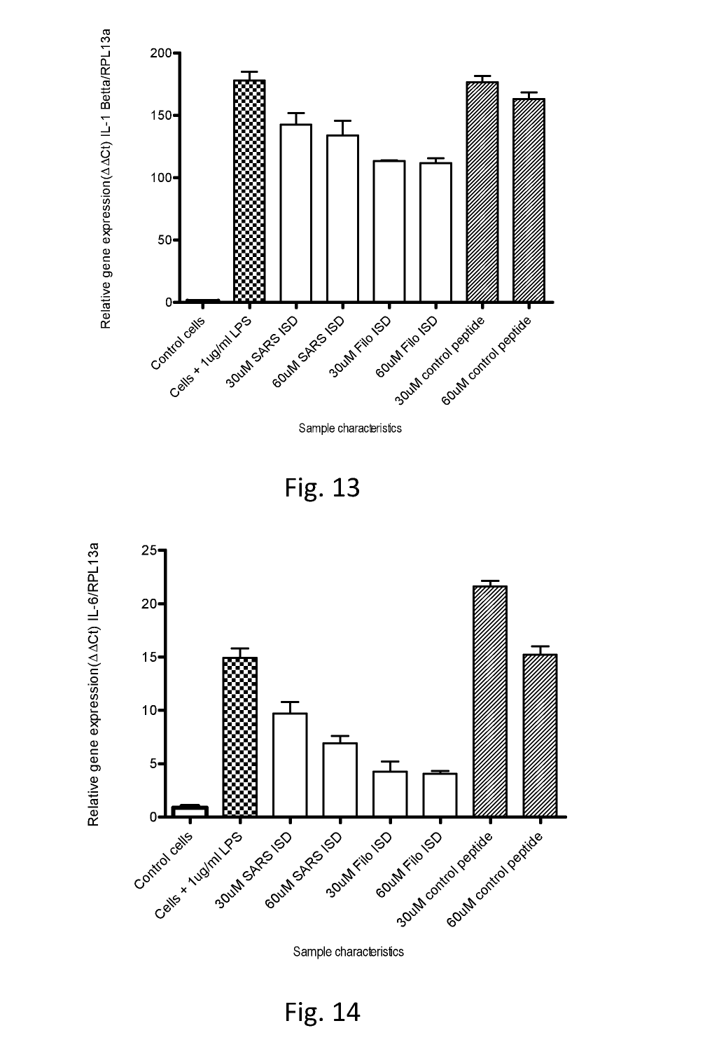

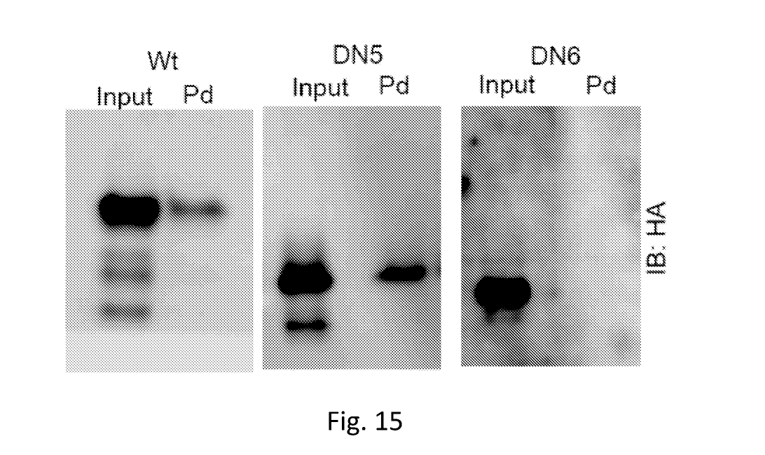

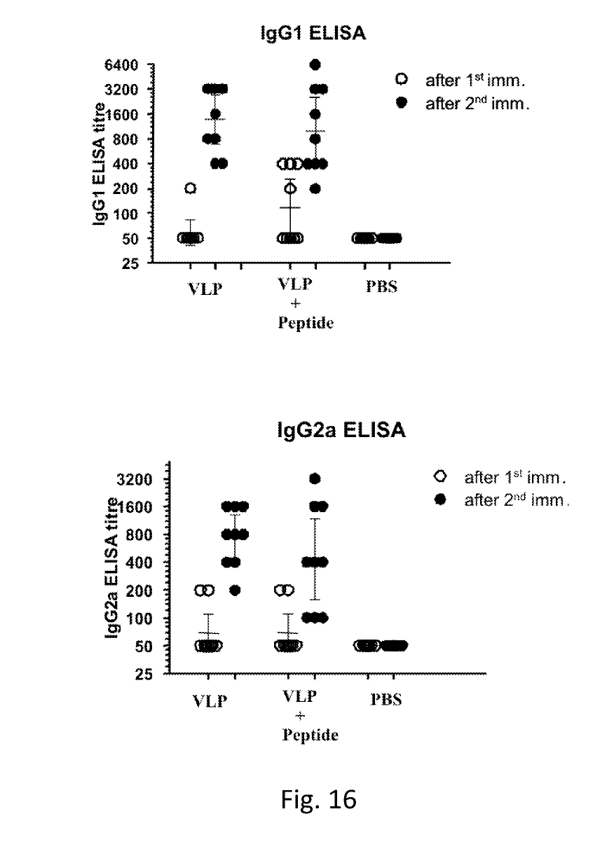

Inactivated A/Vietnam/1203/04 (H5N1) 5/3 reassortant or A/Mississipi/81/1 (H3N2) virus (Institute od Virology, Bratislava, Slovakia) adjusted to 20 HAU/100 μl coating carbonate buffer (pH 9.6) were used as coating antigens. Serial 2-fold dilutions of individual mouse sera, in PBS containing 0.5% i-block (Tropix) were added to the coated plates, and the mixtures incubated for 1.5 hrs at room temperature. Bound antibodies were detected with goat anti-mouse IgG1 and IgG2a conjugated with horseradish peroxidase (Invitrogen). Plates were stained with TMB (KPL) as a substrate and the reaction stopped with H₂SO₄, and the absorbance was measured (wavelength, 450 nm). To determine serum IgG1 or IgG2a titres a cut-off value was defined as mean absorption value of negative control sera+3SD or a cut-off value of 0.1 if values of negative control sera+3SD were still <0.1.

IgG1 and IgG2a ELISA. Baseline serum IgG1 and IgG2a titres were <100 before immunisation. The highest serum IgG1 titres after first immunisation were determined in mice receiving wt VLP and monomeric INF F#2 C17G adjuvant (4/9) whereas only 1 out of 9 animals receiving wt VLPs alone responded to priming. After the second immunisation titres increased in both groups except the control group (PBS). No significant differences were found between groups after 2^{nd} immunisation.

Only few mice (2/9) developed IgG2a titres in response to priming. Following the booster immunization titres markedly increased in all groups except the control group. No significant differences in IgG2a titres were found between adjuvated and non adjuvated groups after 2^{nd} immunisation.

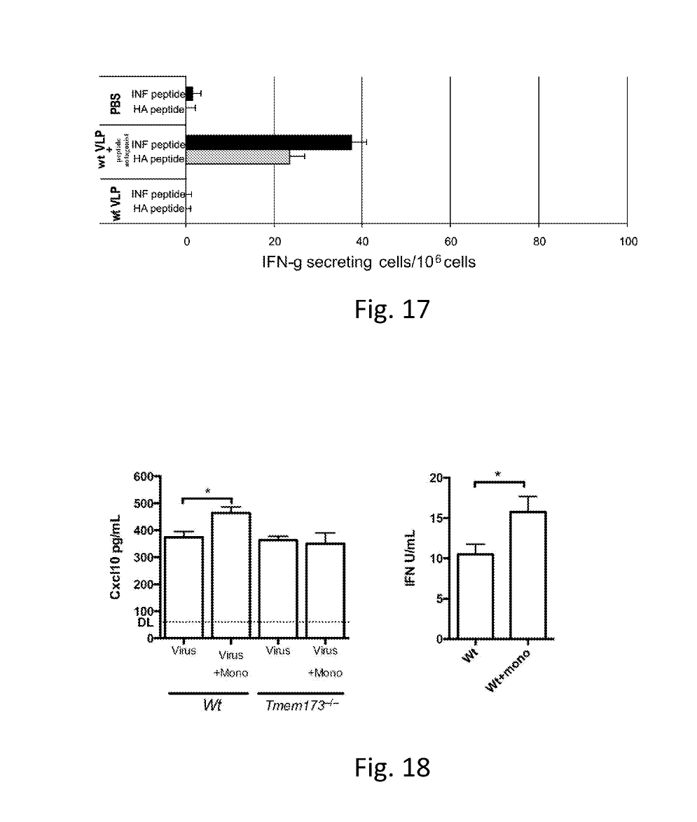

FIG. 17 IFN-γ ELISPOT assay

An immediate ex vivo CD8+ gamma IFN (IFN-γ) enzyme-linked immunospot (ELISPOT) assay was performed utilizing the synthetic peptide (H-2Dd) YSTVASSL and the sponsor's defined epitope marked as INF, both MHC class I H-2Db-restricted immunodominant CTL epitope of influenza A H5N1 virus HA. Briefly, at first, two dilutions of splenocytes 2×10⁵, 5×10⁵ and later 1×10⁵ cells/well (this cell concentration was tested after thawing of splenocyte cultures) were transferred to wells coated with anti-IFN-γ monoclonal antibody. Cells were incubated for 24 h at 37° C. and 5% CO2 in DMEM containing 10% fetal calf serum, penicillin, streptomycin, and 50 μM 2-mercaptoethanol in the presence of the peptide (10 μM for fresh and 20 μM for the thawed spleniocytes). A biotinylated anti IFN-γ MAb (Eubioscience kit) was utilized as a conjugate antibody, followed by incubation of plates with streptavidin peroxidase (Eubioscience kit). Spots representing IFN-γ-secreting CD8+ cells were developed utilizing the substrate 3-amino-9-ethylcarbazole (Sigma) in the presence of hydrogen peroxide in 0.1 M sodium acetate, pH 5.0. The spots were counted with the help of a dissecting microscope, and the results were expressed as the mean number of IFN-γ-secreting cells per 10⁶ cells±standard error of mean (SEM) of duplicate cultures from at least one cell dilution. As controls cells were incubated in the absence of the synthetic peptide or the presence of an irrelevant peptide (ASNENMETM).

IFN-γ secreting CD8+ T cells: About 25 IFN-γ secreting cells could be determined after subtraction of background spots in YSTVASSL—restimulated splenocytes derived from mice immunized with wt VLPs+INF peptide adjuvant. Slightly higher numbers were obtained when the monomeric INF F#2 C17G was used for restimulation.

No significant IFN-γ secreting cells could be detected in non-adjuvated groups tested by IFN-γ ELISPOT.

FIG. 18: Wt BMDCs or STING deficient BMDCs (Tmem173−/−) were infected with Influenza A virus. 30 minutes before Influenza infection BMDCs were pretreated with monomeric INF F#2 C17G. After 18 hours supernatants were analyzed for IFN by bioassay or for the IFN induced gene cxcl10 by ELISA.

The data show that the monomeric INF F#2 C17G (GLFGAIAGFIENGWEGGGGEKEKEK) enhances the interferon response to influenza infection in vitro.

### Envisaged Uses in Vaccines

Known vaccine compositions may be combined with adjuvants of the invention. The following examples, A, B, and C, show examples of vaccines for which the inventors envisage adjuvants of the invention may be used and/or added.

### Example A

### Thiomersal-Reduced Vaccine

### Preparation of Influenza Virus Antigen Preparation Using α-Tocopherol Succinate as a Stabiliser for a Preservative-Free Vaccine

Monovalent split vaccine is prepared according to the following procedure.

Preparation of virus inoculums: On the day of inoculation of embryonated eggs a fresh inoculum is prepared by mixing the working seed lot with a phosphate buffered saline containing gentamycin sulphate at 0.5 mg/ml and hydrocortisone at 25 μg/ml. (virus strain-dependent). The virus inoculum is kept at 2-8° C.

Inoculation of embryonated eggs: Nine to eleven day old embryonated eggs are used for virus replication. Shells are decontaminated. The eggs are inoculated with 0.2 ml of the virus inoculum. The inoculated eggs are incubated at the appropriate temperature (virus strain-dependent) for 48 to 96 hours. At the end of the incubation period, the embryos are killed by cooling and the eggs are stored for 12-60 hours at 2-8° C.

Harvest: The allantoic fluid from the chilled embryonated eggs is harvested. Usually, 8 to 10 ml of crude allantoic fluid is collected per egg.

Concentration and Purification of Whole Virus from Allantoic Fluid:

1. Clarification: The harvested allantoic fluid is clarified by moderate speed centrifugation (range: 4000-14000 g).

2. Adsorption step: To obtain a CaHPO₄ gel in the clarified virus pool, 0.5 mol/L Na₂HPO₄ and 0.5 mol/L CaCl₂ solutions are added to reach a final concentration of CaHPO₄ of 1.5 g to 3.5 g CaHPO/litre depending on the virus strain.

After sedimentation for at last 8 hours, the supernatant is removed and the sediment containing the influenza virus is resolubilised by addition of a 0.26 mol/L EDTA-Na₂ solution, dependent on the amount of CaHPO₄ used.

3. Filtration: The resuspended sediment is filtered on a 6 μm filter membrane.

4. Sucrose gradient centrifugation: The influenza virus is concentrated by isopycnic centrifugation in a linear sucrose gradient (0.55% (w/v)) containing 100 μg/ml Thiomersal. The flow rate is 8-15 litres/hour.

At the end of the centrifugation, the content of the rotor is recovered by four different fractions (the sucrose is measured in a refractometer): fraction 1 55-52% sucrose-fraction 2 approximately 52-38% sucrose fraction 3 38-20% sucrose*fraction 4 20-0% sucrose*virus strain-dependent: fraction 3 can be reduced to 15% sucrose.

For further vaccine preparation, only fractions 2 and 3 are used.

Fraction 3 is washed by diafiltration with phosphate buffer in order to reduce the sucrose content to approximately below 6%. The influenza virus present in this diluted fraction is pelleted to remove soluble contaminants.

The pellet is resuspended and thoroughly mixed to obtain a homogeneous suspension. Fraction 2 and the resuspended pellet of fraction 3 are pooled and phosphate buffer is added to obtain a volume of approximately 40 litres. This product is the monovalent whole virus concentrate.

5. Sucrose gradient centrifugation with sodium deoxycholate: The monovalent whole influenza virus concentrate is applied to a ENI-Mark II ultracentrifuge. The K3 rotor contains a linear sucrose gradient (0.55% (w/v)) where a sodium deoxycholate gradient is additionally overlayed. Tween 80 is present during splitting up to 0.1% (w/v) and Tocopherol succinate is added for B-strain-viruses up to 0.5 mM. The maximal sodium deoxycholate concentration is 0.7-1.5% (w/v) and is strain dependent. The flow rate is 8-15 litres/hour.

At the end of the centrifugation, the content of the rotor is recovered by three different fractions (the sucrose is measured in a refractometer) Fraction 2 is used for further processing. Sucrose content for fraction limits (47-18%) varies according to strains and is fixed after evaluation:

6. Sterile filtration: The split virus fraction is filtered on filter membranes ending with a 0.2 μm membrane. Phosphate buffer containing 0.025% (w/v) Tween 80 and (for B strain viruses) 0.5 mM Tocopherol succinate is used for dilution. The final volume of the filtered fraction 2 is 5 times the original fraction volume.

7. Inactivation: The filtered monovalent material is incubated at 22±2° C. for at most 84 hours (dependent on the virus strains, this incubation can be shortened). Phosphate buffer containing 0.025% (w/v). Tween 80 is then added in order to reduce the total protein content down to max. 250 μg/ml. For B strain viruses, a phosphate buffered saline containing 0.025% (w/v) Tween 80 and 0.25 mM Tocopherol succinate is applied for dilution to reduce the total protein content down to 250 μg/ml. Formaldehyde is added to a final concentration of 50 μg/ml and the inactivation takes place at 20° C.±2° C. for at least 72 hours.

8. Ultrafiltration: The inactivated split virus material is concentrated at least 2 fold in a ultrafiltration unit, equipped with cellulose acetate membranes with 20 kDa MWCO. The Material is subsequently washed with phosphate buffer containing 0.025% (w/v) Tween 80 and following with phosphate buffered saline containing 0.01% (w/v) Tween. For B strain virus a phosphate buffered saline containing 0.01% (w/v) Tween 80 and 0.1 mM Tocopherol succinate is used for washing.

9. Final sterile filtration: The material after ultrafiltration is filtered on filter membranes ending with a 0.2 μm membrane. Filter membranes are rinsed and the material is diluted if necessary such that the protein concentration does not exceed 500 μg/ml with phosphate buffered saline containing 0.01% (w/v) Tween 80 and (for B strain viruses) 0.1 mM Tocopherol succinate.

10. Storage: The monovalent final bulk is stored at 2-8° C. for a maximum of 18 months.

### Example B

### General Method for Production of a Hemagglutinin Based Influenza Vaccine

The recombinant HA vaccines contains full length uncleaved HA (HAO) glycoprotein from the influenza A/Beijing/32/92 (H3N2) virus. Recombinant HAO (rHAO) are produced in cultures of Lepidopteran (insect) cells following exposure to a baculovirus vector containing cDNA inserts encoding the HA gene. The expressed protein is purified under non-denaturing conditions to >95%, as measured by quantitative scanning densitometry of the bulk antigen electrophoresed on sodium dodecyl sulfate-polyacrylamide gels. The identity of the peptide is confirmed by amino acid analysis, N-terminal sequencing and Western blot analysis with antiinfluenza A/Beijing/32/92 sera. The rHAO vaccines contains a specified amount of the synthetic HA antigen either dissolved in a phosphate-buffered saline solution or adsorbed to aluminum phosphate (alum) adjuvant in the form of a gel suspension.

### Example C

### Recombinant Protein Vaccine

### Preparation of HBV Vaccine

1.1. Preparation of Recombinant Entire Surface Antigen (preS and S Antigens; L-HBsAg)

### (I)-I Cloning

PCR is performed using a vector containing HBV genome (HBV315, Korean Biochem. J. 17: 70-79, 1984) as a template to amplify a coding region of envelopee gene (preSI-preS2-S) and an entire 3′-UTR containing polyadenylation site, and then introduced into an expression vector. At this time, PCR is performed using a Pfu DNA polymerase, and primers are prepared to amplify the coding region of HBsAg and the entire 3′-UTR (forward primer: 5-GGA AGA TCT CAA TCT CGG GAA-3, reverse primer: 5-GGA AGA TCT CGA ATA GAA GGA AAG-3). A PCR product of about 2.75 kbp is obtained, and ligated with a pMSG vector (see Korean Patent Application No. 10-2000-0043996 and PCT/KROI/01285) which is linearized with BgIII enzyme. CHO cells are transformed with the vector to give transformants, and Western blot is performed to confirm the expression of entire surface antigen (L-HBsAg), followed by screening transformants for high-level expression. The selected transformants is designated as CHO DG44/L-HBsAg(J2.1)-GIOI.

### (I)-2 Establishment of Cell Line in Suspension Culture

The selected cell line (5×10 cells) is inoculated in a T-175 flask. The cell line is cultured in media containing 10% serum, and the attached cells are treated with 0.25% trypsin. Then, the cells are centrifuged at 1200 rpm for 5 min to remove the residual trypsin. The single cells are resuspended in protein-free media (HyQ SFM4CH0, Hyclone), inoculated in 250 ml spinner flasks with 100 ml working volume, and cultured at 80 rpm and 37° C. The cells are inoculated at the initial concentration of 5×10 cells/ml. When the concentration of the cells approaches 1.5×10 cells/ml, the cells are continuously subcultured using the same initial concentration. Finally, the cell lines adapted to suspension culture are obtained.

### (2) Culture

Cell inoculation is prepared by subculturing from MCB (Master Cell Bank). At this time, serum-free media (HyQ SFM4CHO, Hyclone) are used as a basic medium, and the cells are inoculated at the concentration of 5×10 cells/ml in 250 ml spinner flasks and cultured at 34° C. and 80 rpm. After three days, the cells are subcultured in 1 L Spinner flasks to expand the number of cells. Then, the cells are inoculated in a 7.5 L bioreactor, and cultured at pH 7.2, 34° C. and at the stirring speed of 80 rpm. After three days, citric acid and HyQ LSIOOO are added, and the cells are cultured for another three days.

### (3) Purification

The culture media recovered from the bioreactor are centrifuged to remove cell debris and passed through a 0.45 um filter to remove impurities. The expressed HBV surface antigen is purified by an equilibrated phenyl-sepharose chromatography, DEAE-sepharose chromatography, and sepharose 4 FF chromatography.

The purified LHBsAg may be used as a vaccine by itself or combined with an adjuvant.

1.-61. (canceled)

62. A vaccine composition comprising an immunosuppressive domain and a vaccine antigen, wherein the immunosuppressive domain serves as an adjuvant.

63. The vaccine composition according to claim 62, wherein said vaccine is for the treatment or prophylaxis of a virus infection.

64. The vaccine composition according to claim 63, wherein said virus infection is caused by an influenza virus.

65. The vaccine composition according to claim 62, wherein said immunosuppressive domain is from a virus.

66. The vaccine composition according to claim 65, wherein said immunosuppressive domain is from an influenza virus.

67. The vaccine composition according to claim 62, wherein said vaccine composition is for preventing a virus infection, and wherein said virus infection and said immunosuppressive domain are from the same genus of virus.

68. The vaccine composition according to claim 67, wherein said virus infection and said immunosuppressive domain are from the same species of virus.

69. The vaccine composition according to claim 62, wherein said vaccine composition is for influenza and comprises an influenza antigen and a peptide which forms part of an immunosuppressive domain of an influenza virus.

70. The vaccine composition according to claim 69, wherein said antigen and said immunosuppressive domain are from the same clade or strain of influenza virus.

71. A vaccine composition comprising a vaccine antigen and a peptide, said peptide serving as an adjuvant and comprising a mutated form or a non-mutated form of an immunosuppressive domain.

72. The vaccine composition according to claim 71 comprising said mutated form, wherein said mutated form comprises 1, 2, 3 or 4 mutations, deletions or insertions with respect to said non-mutated form.

73. The vaccine composition according to claim 71, wherein said peptide forms part of a surface protein of a pathogen.

74. The vaccine composition according to claim 73, wherein said peptide forms part of a surface protein of a virus.

75. The vaccine composition according to claim 74, wherein said peptide forms part of an enveloped virus surface glycoprotein.

76. The vaccine composition according to claim 71, wherein said peptide has a length of 8-18 amino acids.

77. The vaccine composition according to claim 71, wherein said peptide has a length of 5-200 amino acids.

78. The vaccine composition according to claim 71, further comprising a fusion peptide from a fusion protein.

79. The vaccine composition according to claim 78, wherein the fusion protein is from an enveloped virus.

80. The vaccine composition according to claim 78, wherein the fusion protein is a type I fusion protein.

81. The vaccine composition according to claim 78, wherein the fusion protein is a type II fusion protein.

82. The vaccine composition according to claim 78, wherein said fusion peptide has 1, 2, 3 or 4 mutations, deletions or insertions with respect to a wild type of the fusion peptide.

83. The vaccine composition according to claim 71, wherein said peptide, or a functional homologue thereof, binds to a STING complex.

84. The vaccine composition according to claim 71, wherein said peptide, or a functional homologue thereof, affects type I interferon responses.

85. The vaccine composition according to claim 84, wherein said type I interferon responses are induced by membrane fusion.

86. The vaccine composition according to claim 71, wherein said peptide comprises an entire sequence selected from Table 1 or selected from SEQ ID NOS: 1 to 281.

87. The vaccine composition according to claim 71, wherein said peptide comprises the sequence of SEQ ID NO: 275 or INF ISD C17G (SEQ ID NO: 281).

88. The vaccine composition according to claim 71, wherein said peptide has immunosuppressive activity as a dimer or a multimer, or when coupled to a carrier protein.

89. The vaccine composition according to claim 71, wherein said peptide has no or diminished immunosuppressive activity as a monomer while having immunosuppressive activity in a dimeric form.

90. The vaccine composition according to claim 71, wherein said peptide is attached to at least one biological membrane.

91. A method of immunizing a subject, the method comprising the step of administering to the subject a vaccine composition according to claim 71.


SEQUENCE LISTING (281 sequences)

SEQ ID NO: 1 - protein, 27 aa, Vesiculovirus
VQVTPHHVLVDEYTGEWVDSQFINGKC

SEQ ID NO: 2 - protein, 14 aa, flavivirus
DRGWGNGCGLFGKG

SEQ ID NO: 3 - protein, 17 aa, Hepatitis C virus
GLIHLHQNIVDVQYLYG

SEQ ID NO: 4 - protein, 16 aa, Influenza A and B virus
GLFGAIAGFIENGWEG

SEQ ID NO: 5 - protein, 22 aa, Rabies virus
GFTCTGVVTEAETYTNFVGYVT

SEQ ID NO: 6 - protein, 18 aa, Rabies virus
SLHNPYPDYRWLRTVKTT

SEQ ID NO: 7 - protein, 19 aa, Aroa virus
GDAAWDFGSVGGVFNSLGK

SEQ ID NO: 8 - protein, 19 aa, Dengue
GGTAWDFGSIGGVFTSVGK

SEQ ID NO: 9 - protein, 19 aa, Dengue
GDTAWDFGSLGGVFTSVGK

SEQ ID NO: 10 - protein, 19 aa, Dengue
GETAWDFGSVGGLLTSLGK

SEQ ID NO: 11 - protein, 19 aa, Japanese encephalitis virus
LGDTAWDFGSIGGVFNSIG

SEQ ID NO: 12 - protein, 19 aa, Koutango virus
LGDTAWDFGSVGGIFTSLG

SEQ ID NO: 13 - protein, 19 aa, Murray Valley encephalitis virus
LGDTAWDFGSVGGVFNSIG

SEQ ID NO: 14 - protein, 19 aa, Usutu virus
LGDTAWDFGSVGGIFNSVG

SEQ ID NO: 15 - protein, 19 aa, West Nile virus
LGDTAWDFGSVGGVFTSVG

SEQ ID NO: 16 - protein, 19 aa, Kokobera virus
IGDDAWDFGSVGGILNSVG

SEQ ID NO: 17 - protein, 19 aa, Modoc virus
VGSAFWNSDQRFSAINLMD

SEQ ID NO: 18 - protein, 14 aa, Modoc virus
DRGWGNGCALFGKG

SEQ ID NO: 19 - protein, 19 aa, Sepik virus
TGEHSWDFGSTGGFFASVG

SEQ ID NO: 20 - protein, 19 aa, Bagaza virus
LGDTAWDFGSVGGFFTSLG

SEQ ID NO: 21 - protein, 19 aa, Yokose virus
IGDDAWDFGSTGGIFNTIG

SEQ ID NO: 22 - protein, 20 aa, Apoi virus
SSAFWNSDEPFHFSNLISII

SEQ ID NO: 23 - protein, 20 aa, Entebbe bat virus
GDDAWDFGSTGGIFNTIGKA

SEQ ID NO: 24 - protein, 20 aa, Rio Bravo virus
SSAYWSSSEPFTSAGIMRIL

SEQ ID NO: 25 - protein, 20 aa, Saboya virus
GSSSWDFSSAGGFFGSIGKA

SEQ ID NO: 26 - protein, 20 aa, Meaban virus
GDAAWDFGSVGGFMTSIGRA

SEQ ID NO: 27 - protein, 14 aa, Meaban virus
DRGWGNHCGLFGKG

SEQ ID NO: 28 - protein, 20 aa, Saumarez Reef virus
GETAWDFGSAGGFFTSVGRG

SEQ ID NO: 29 - protein, 20 aa, Tyuleniy virus
GEAAWDFGSAGGFFQSVGRG

SEQ ID NO: 30 - protein, 20 aa, Zika virus
LGDTAWDFGSVGGVFNSLGK

SEQ ID NO: 31 - protein, 19 aa, Kyasanur forest disease virus
VGEHAWDFGSVGGMLSSVG

SEQ ID NO: 32 - protein, 20 aa, Langat virus
VLGEHAWDFGSVGGVMTSIG

SEQ ID NO: 33 - protein, 19 aa, Louping ill virus
IGEHAWDFGSAGGFFSSIG

SEQ ID NO: 34 - protein, 19 aa, Omsk hemorrhagic fever virus
LGEHAWDFGSTGGFLSSIG

SEQ ID NO: 35 - protein, 19 aa, Powassan virus
VGEHAWDFGSVGGILSSVG

SEQ ID NO: 36 - protein, 14 aa, Powassan virus
DRGWGNHCGFFGKG

SEQ ID NO: 37 - protein, 19 aa, Tick-borne encephalitis virus
IGEHAWDFGSAGGFLSSIG

SEQ ID NO: 38 - protein, 19 aa, Tick-borne encephalitis virus
IGEHAWDFGSTGGFLTSVG

SEQ ID NO: 39 - protein, 19 aa, Tick-borne encephalitis virus
IGEHAWDFGSTGGFLASVG

SEQ ID NO: 40 - protein, 19 aa, Yaounde virus
LGDTAWDFGSIGGVFTSLG

SEQ ID NO: 41 - protein, 19 aa, Banzi virus
VGSSSWDFSSTSGFFSSVG

SEQ ID NO: 42 - protein, 19 aa, Bouboui virus
VGRSSWDFSSAGGFFSSVG

SEQ ID NO: 43 - protein, 19 aa, Yellow fever virus
MGDTAWDFSSAGGFFTSVG

SEQ ID NO: 44 - protein, 14 aa, Unclassified Flavivirus and culex flavivirus
NRGWGTGCFKWGIG

SEQ ID NO: 45 - protein, 14 aa, Unclassified Flavivirus and aedes flavirus
NRGWGTGCFEWGLG

SEQ ID NO: 46 - protein, 23 aa, Aedes flavivirus
HVAGRYSKHGMAGIGSVWEDLVR

SEQ ID NO: 47 - protein, 14 aa, Culex flavivirus
VDKYRRFGTAGVGG

SEQ ID NO: 48 - protein, 17 aa, Hepatitis C virus
GLIHLHRNIVDVQYLYG

SEQ ID NO: 49 - protein, 17 aa, Hepatitis C virus
GLIHLHQNIVDVQYMYG

SEQ ID NO: 50 - protein, 17 aa, Hepatitis C virus
GLIHLHQNIVDTQYLYG

SEQ ID NO: 51 - protein, 24 aa, Border disease virus
NTTLLNGSAFQLICPYGWVGRVEC

SEQ ID NO: 52 - protein, 19 aa, Border disease virus
SYFQQYMLKGQYQYWFDLE

SEQ ID NO: 53 - protein, 24 aa, Bovine viral diarrhea virus
NTTLLNGPAFQMVCPLGWTGTVSC

SEQ ID NO: 54 - protein, 19 aa, Bovine viral diarrhea virus
SYFQQYMLKGEYQYWFDLE

SEQ ID NO: 55 - protein, 22 aa, Bovine viral diarrhea virus
SLLNGPAFQMVCPQGWTGTIEC

SEQ ID NO: 56 - protein, 20 aa, Bovine viral diarrhea virus
DRYFQQYMLKGKWQYWFDLD

SEQ ID NO: 57 - protein, 22 aa, Classical swine fever virus
TLLNGSAFYLVCPIGWTGVIEC

SEQ ID NO: 58 - protein, 19 aa, Classical swine fever virus
SYFQQYMLKGEYQYWFDLD

SEQ ID NO: 59 - protein, 22 aa, Bovine viral diarrhea virus
TLLNGPAFQLVCPYGWTGTIEC

SEQ ID NO: 60 - protein, 23 aa, Bovine viral diarrhea virus
DNYFQQYMLKGKYQYWFDLEATD

SEQ ID NO: 61 - protein, 22 aa, Chamois pestivirus
TLLNGSAFQMVCPFGWTGQVEC

SEQ ID NO: 62 - protein, 23 aa, Chamois pestivirus
DSYFQQYMLKGEYQYWFDLDAKD

SEQ ID NO: 63 - protein, 22 aa, Alpha-virus
GVYPFMWGGAYCFCDTENTQVS

SEQ ID NO: 64 - protein, 23 aa, Alpha-virus
APFGCEIYTNPIRAENCAVGSIP

SEQ ID NO: 65 - protein, 22 aa, Alpha-virus
SDFGGIATVKYSASKSGKCAVH

SEQ ID NO: 66 - protein, 28 aa, Alpha-virus
FSTANIHPEFRLQICTSYVTCKGDCHPP

SEQ ID NO: 67 - protein, 15 aa, Venezuelan equine encephalitis virus
GVYPFMWGGAYCFCD

SEQ ID NO: 68 - protein, 19 aa, Venezuelan equine encephalitis virus
GDCHPPKDHIVTHPQYHAQ

SEQ ID NO: 69 - protein, 12 aa, Venezuelan equine encephalitis virus
AVSKTAWTWLTS

SEQ ID NO: 70 - protein, 17 aa, Chikungunya virus
VHCAAECHPPKDHIVNY

SEQ ID NO: 71 - protein, 20 aa, Chikungunya virus
PASHTTLGVQDISATAMSWV

SEQ ID NO: 72 - protein, 30 aa, Rubella virus
ACTFWAVNAYSSGGYAQLASYFNPGGSYYK

SEQ ID NO: 73 - protein, 27 aa, Rubella virus
QYHPTACEVEPAFGHSDAACWGFPTDT

SEQ ID NO: 74 - protein, 23 aa, Rubella virus
MSVFALASYVQHPHKTVRVKFHT

SEQ ID NO: 75 - protein, 36 aa, Human respiratory syncytial virus
GIIRTLPDGCHYISNKGVDRVQVGNTVYYLSKEVGK

SEQ ID NO: 76 - protein, 22 aa, Rubella virus
NVTTEHPFCNMPHGQLEVQVPP

SEQ ID NO: 77 - protein, 20 aa, Rubella virus
DPGDLVEYIMNYTGNQQSRW

SEQ ID NO: 78 - protein, 23 aa, Rubella virus
GSPNCHGPDWASPVCQRHSPDCS

SEQ ID NO: 79 - protein, 15 aa, Rubella virus
RLVGATPERPRLRLV

SEQ ID NO: 80 - protein, 13 aa, Rubella virus
DADDPLLRTAPGP

SEQ ID NO: 81 - protein, 18 aa, Rubella virus
GEVWVTPVIGSQARKCGL

SEQ ID NO: 82 - protein, 17 aa, Rabies virus
QHMELLESSVIPLVHPL

SEQ ID NO: 83 - protein, 19 aa, Tembusu virus
LGDTAWDFGSVGGVLTSIG

SEQ ID NO: 84 - protein, 19 aa, Ilheus virus
LGDTAWDFGSVGGIFNSIG

SEQ ID NO: 85 - protein, 14 aa, Aroa virus
NRGWNNGCGLFGKG

SEQ ID NO: 86 - protein, 14 aa, Rubella virus
HIRAGPYGHATVEM

SEQ ID NO: 87 - protein, 15 aa, Rubella virus
PEWIHAHTTSDPWHP

SEQ ID NO: 88 - protein, 18 aa, Rubella virus
PGPLGLKFKTVRPVALPR

SEQ ID NO: 89 - protein, 21 aa, Rubella virus
ALAPPRNVRVTGCYQCGTPAL

SEQ ID NO: 90 - protein, 20 aa, Rubella virus
EGLAPGGGNCHLTVNGEDVG

SEQ ID NO: 91 - protein, 17 aa, Rabies virus
CKLKLCGVLGLRLMDGT

SEQ ID NO: 92 - protein, 23 aa, Rubella virus
RASARVIDPAAQSFTGVVYGTHT

SEQ ID NO: 93 - protein, 24 aa, Hanta-virus
TAVSETRQTWAEWAAAHWWQLTLG

SEQ ID NO: 94 - protein, 20 aa, Hanta-virus
NPPDCPGVGTGCTACGVYLD

SEQ ID NO: 95 - protein, 21 aa, Hanta-virus
RKVCIQLGTEQTCKTIDSNDC

SEQ ID NO: 96 - protein, 33 aa, Hanta-virus
DTLLFLGPLEEGGMIFKQWCTTTCQFGDPGDIM

SEQ ID NO: 97 - protein, 24 aa, Hanta-virus
GSFRKKCSFATLPSCQYDGNTVSG

SEQ ID NO: 98 - protein, 15 aa, Hanta-virus
ATKDSFQSFNITEPH

SEQ ID NO: 99 - protein, 17 aa, Hanta-virus
GSGVGFNLVCSVSLTEC

SEQ ID NO: 100 - protein, 22 aa, Hanta-virus
KACDSAMCYGSSTANLVRGQNT

SEQ ID NO: 101 - protein, 30 aa, Hanta-virus
GKGGHSGSKFMCCHDKKCSATGLVAAAPHL

SEQ ID NO: 102 - protein, 19 aa, Hanta-virus
DDGAPQCGVHCWFKKSGEW

SEQ ID NO: 103 - protein, 22 aa, Ortho-bunya-virus
KHDELCTGPCPVNINHQTGWLT

SEQ ID NO: 104 - protein, 22 aa, Ortho-bunya-virus
WGCEEFGCLAVSDGCVFGSCQD

SEQ ID NO: 105 - protein, 24 aa, Ortho-bunya-virus
GNGVPRFDYLCHLASRKEVIVRKC

SEQ ID NO: 106 - protein, 14 aa, Ortho-bunya-virus
SCAGCINCFQNIHC

SEQ ID NO: 107 - protein, 17 aa, Rift Valley fever virus
KTVSSELSCREGQSYWT

SEQ ID NO: 108 - protein, 13 aa, Rift Valley fever virus
GSFSPKCLSSRRC

SEQ ID NO: 109 - protein, 27 aa, Rift Valley fever virus
ENKCFEQCGGWGCGCFNVNPSCLFVHT

SEQ ID NO: 110 - protein, 21 aa, Rift Valley fever virus
WGSVSLSLDAEGISGSNSFSF

SEQ ID NO: 111 - protein, 13 aa, Rift Valley fever virus
RQGFLGEIRCNSE

SEQ ID NO: 112 - protein, 23 aa, Rift Valley fever virus
AHESCLRAPNLVSYKPMIDQLEC

SEQ ID NO: 113 - protein, 15 aa, Rift Valley fever virus
DPFVVFERGSLPQTR

SEQ ID NO: 114 - protein, 17 aa, Rift Valley fever virus
QAFSKGSVQADLTLMFD

SEQ ID NO: 115 - protein, 17 aa, Rift Valley fever virus
CDAAFLNLTGCYSCNAG

SEQ ID NO: 116 - protein, 19 aa, Rift Valley fever virus
CQILHFTVPEVEEEFMYSC

SEQ ID NO: 117 - protein, 12 aa, Rift Valley fever virus
STVVNPKSGSWN

SEQ ID NO: 118 - protein, 17 aa, Rift Valley fever virus
FFDWFSGLMSWFGGPLK

SEQ ID NO: 119 - protein, 16 aa, Influenza A virus
GLFGAIAGFIEGGWTG

SEQ ID NO: 120 - protein, 16 aa, Influenza A virus
GLFGAIAGFIEGGWQG

SEQ ID NO: 121 - protein, 16 aa, Influenza A virus
GIFGAIAGFIENGWEG

SEQ ID NO: 122 - protein, 16 aa, Influenza A virus
GLFGAIAGFIENGWQG

SEQ ID NO: 123 - protein, 16 aa, Influenza A virus
GIFGAIAGFIEGGWTG

SEQ ID NO: 124 - protein, 16 aa, Influenza A virus
GLFGAIAGFIEGGWSG

SEQ ID NO: 125 - protein, 16 aa, Influenza A virus
GLFGAIAGFIEGGWPG

SEQ ID NO: 126 - protein, 16 aa, Influenza A virus
GFFGAIAGFLEGGWEG

SEQ ID NO: 127 - protein, 16 aa, Avulavirus
GAIALGVATAAAVTAG

SEQ ID NO: 128 - protein, 17 aa, Human respiratory syncytial virus
FLGLILGLGAAVTAGVA

SEQ ID NO: 129 - protein, 15 aa, Human respiratory syncytial virus
TNEAVVSLTNGMSVL

SEQ ID NO: 130 - protein, 13 aa, Human respiratory syncytial virus
VIRFQQLNKRLLE

SEQ ID NO: 131 - protein, 10 aa, Human respiratory syncytial virus
REFSSNAGLT

SEQ ID NO: 132 - protein, 14 aa, Human respiratory syncytial virus
MLTDRELTSIVGGM

SEQ ID NO: 133 - protein, 16 aa, Human respiratory syncytial virus
YVIQLPLFGVMDTDCW

SEQ ID NO: 134 - protein, 21 aa, Human respiratory syncytial virus
CLARADNGWYCHNAGSLSYFP

SEQ ID NO: 135 - protein, 16 aa, Human respiratory syncytial virus
DTLKSLTVPVTSRECN

SEQ ID NO: 136 - protein, 21 aa, Human respiratory syncytial virus
YDCKISTSKTYVSTAVLTTMG

SEQ ID NO: 137 - protein, 13 aa, Human respiratory syncytial virus
VSCYGHNSCTVIN

SEQ ID NO: 138 - protein, 22 aa, Rabies virus
ESLVIISPSVADLDPYDRSLHS

SEQ ID NO: 139 - protein, 34 aa, Human respiratory syncytial virus
PLSFPDDKFDVAIRDVEHSINQTRTFLKASDQLL

SEQ ID NO: 140 - protein, 25 aa, Human respiratory syncytial virus
KIMTSKTDISSSVITSIGAIVSCYG

SEQ ID NO: 141 - protein, 18 aa, Coronavirinae
RSAIEDLLFDKVKLSDVG

SEQ ID NO: 142 - protein, 17 aa, Coronavirinae
VPFYLNVQYRINGLGVT

SEQ ID NO: 143 - protein, 21 aa, Coronavirinae
VLSQNQKLIANAFNNALHAIQ

SEQ ID NO: 144 - protein, 16 aa, Coronavirinae
TNSALVKIQAVVNANA

SEQ ID NO: 145 - protein, 24 aa, Coronavirinae
AEAQIDRLINGRLTALNAYVSQQL

SEQ ID NO: 146 - protein, 31 aa, Coronavirinae
SAAQAMEKVNECVKSQSSRINFCGNGNHIIS

SEQ ID NO: 147 - protein, 14 aa, Coronavirinae
APYGLYFIHFNYVP

SEQ ID NO: 148 - protein, 32 aa, Coronavirinae
LQEAIKVLNHSYINLKDIGTYEYYVKWPWYVW

SEQ ID NO: 149 - protein, 23 aa, Arena-virus
NALINDQLIMKNHLRDIMGIPYC

SEQ ID NO: 150 - protein, 20 aa, Arena-virus
FTWTLSDSEGKDTPGGYCLT

SEQ ID NO: 151 - protein, 29 aa, Arena-virus
KCFGNTAIAKCNQKHDEEFCDMLRLFDFN

SEQ ID NO: 152 - protein, 24 aa, Arena-virus
MLQKEYMERQGKTPLGLVDLFVFS

SEQ ID NO: 153 - protein, 16 aa, Hepatitis B virus
FNPLGFFPSHQLDPLF

SEQ ID NO: 154 - protein, 13 aa, Hepatitis B virus
ADWDKNPNKDPWP

SEQ ID NO: 155 - protein, 20 aa, Hepatitis B virus
MESITSGFLGPLLVLQAVFF

SEQ ID NO: 156 - protein, 25 aa, Hepatitis B virus
LLTRILTIPQSLDSWWTSLNFLGGA

SEQ ID NO: 157 - protein, 12 aa, Hepatitis B virus
CPPTCPGYRWMC

SEQ ID NO: 158 - protein, 18 aa, Hepatitis B virus
LFILLLCLIFLLVLLDYQ

SEQ ID NO: 159 - protein, 15 aa, Rubella virus
ETRTVWQLSVAGVSC

SEQ ID NO: 160 - protein, 24 aa, Bovine ephemeral fever virus
LDGYLCRKQKWEVTCTETWYFVTD

SEQ ID NO: 161 - protein, 14 aa, Bovine ephemeral fever virus
KYQIIEVIPTENEC

SEQ ID NO: 162 - protein, 24 aa, Bovine ephemeral fever virus
LKGEYIPPYYPPTNCVWNAIDTQE

SEQ ID NO: 163 - protein, 18 aa, Bovine ephemeral fever virus
IEDPVTMTLMDSKFTKPC

SEQ ID NO: 164 - protein, 13 aa, Bovine ephemeral fever virus
LHCQIKSWECIPV

SEQ ID NO: 165 - protein, 15 aa, Bovine ephemeral fever virus
SHRNMMEALYLESPD

SEQ ID NO: 166 - protein, 20 aa, Bovine ephemeral fever virus
LTFCGYNGILLDNGEWWSIY

SEQ ID NO: 167 - protein, 18 aa, Bovine ephemeral fever virus
ELEHEKCLGTLEKLQNGE

SEQ ID NO: 168 - protein, 17 aa, Bovine ephemeral fever virus
LDLSYLSPSNPGKHYAY

SEQ ID NO: 169 - protein, 15 aa, Bovine ephemeral fever virus
IRAVCYYHTFSMNLD

SEQ ID NO: 170 - protein, 20 aa, Vesiculovirus
EWKTTCDYRWYGPQYITHSI

SEQ ID NO: 171 - protein, 15 aa, Vesiculovirus
LGFPPQSCGWASVTT

SEQ ID NO: 172 - protein, 23 aa, Dengue
KGSSIGKMFESTYRGAKRMAILG

SEQ ID NO: 173 - protein, 23 aa, Dengue
KGSSIGKMFEATARGARRMAILG

SEQ ID NO: 174 - protein, 23 aa, Dengue
KGSSIGQMFETTMRGAKRMAILG

SEQ ID NO: 175 - protein, 18 aa, Hepatitis C virus
PALSTGLIHLHQNIVDVQ

SEQ ID NO: 176 - protein, 18 aa, Hepatitis C virus
PALSTGLIHLHRNIVDVQ

SEQ ID NO: 177 - protein, 18 aa, Hepatitis C virus
PALSTGLIHLHQNIVDTQ

SEQ ID NO: 178 - protein, 18 aa, Influenza A virus
WTYNAELLVLLENERTLD

SEQ ID NO: 179 - protein, 18 aa, Influenza A virus
NAELLVLLENERTLDYHD

SEQ ID NO: 180 - protein, 18 aa, Influenza A virus
WTYNAELLVLMENERTLD

SEQ ID NO: 181 - protein, 18 aa, Influenza A virus
NAELLVLMENERTLDYHD

SEQ ID NO: 182 - protein, 18 aa, Influenza A virus
WSYNAELLVALENQHTID

SEQ ID NO: 183 - protein, 18 aa, Influenza A virus
NAELLVALENQHTIDLTD

SEQ ID NO: 184 - protein, 18 aa, Influenza A virus
NAELLVALENQHTIDVTD

SEQ ID NO: 185 - protein, 18 aa, Influenza A virus
NAELLVLMENERTLDFHD

SEQ ID NO: 186 - protein, 18 aa, Influenza A virus
NAELLVLLENERTLDMHD

SEQ ID NO: 187 - protein, 18 aa, Influenza A virus
WSYNAELLVAMENQHTID

SEQ ID NO: 188 - protein, 18 aa, Influenza A virus
NAELLVAMENQHTIDLAD

SEQ ID NO: 189 - protein, 18 aa, Influenza A virus
WAYNAELLVLLENQKTLD

SEQ ID NO: 190 - protein, 18 aa, Influenza A virus
NAELLVLLENQKTLDEHD

SEQ ID NO: 191 - protein, 18 aa, Influenza A virus
WTYQAELLVAMENQHTID

SEQ ID NO: 192 - protein, 18 aa, Influenza A virus
QAELLVAMENQHTIDMAD

SEQ ID NO: 193 - protein, 18 aa, Influenza A virus
WSYNAQLLVLLENEKTLD

SEQ ID NO: 194 - protein, 18 aa, Influenza A virus
NAQLLVLLENEKTLDLHD

SEQ ID NO: 195 - protein, 18 aa, Influenza A virus
WSYNAKLLVLLENDKTLD

SEQ ID NO: 196 - protein, 18 aa, Influenza A virus
NAKLLVLLENDKTLDMHD

SEQ ID NO: 197 - protein, 18 aa, Influenza A virus
WSYNAKLLVLIENDRTLD

SEQ ID NO: 198 - protein, 18 aa, Influenza A virus
NAKLLVLIENDRTLDLHD

SEQ ID NO: 199 - protein, 18 aa, Influenza A virus
ISSQIELAVLLSNEGIIN

SEQ ID NO: 200 - protein, 18 aa, Influenza A virus
QIELAVLLSNEGIINSED

SEQ ID NO: 201 - protein, 15 aa, Influenza virus
GLFGAAGFIENGWEG

SEQ ID NO: 202 - protein, 14 aa, flavivirus
DRGWGNGCGDFGKG

SEQ ID NO: 203 - protein, 18 aa, Influenza virus
NAELLVLLENERTLDFHD

SEQ ID NO: 204 - protein, 19 aa, Dengue
GDTAWDFGSVGGVLNSLGK

SEQ ID NO: 205 - protein, 27 aa, Porcine pestivirus isolate Bungowannah
TLLNGPAFQLVCPYGWTGTIECDSYYQ

SEQ ID NO: 206 - protein, 16 aa, Rabies virus
ILGPDGNVLIPEMQSS

SEQ ID NO: 207 - protein, 14 aa, Rubella virus
LLNTPPPYQVSCGG

SEQ ID NO: 208 - protein, 18 aa, Influenza A virus
WSYNAELLVAMENQHLAD

SEQ ID NO: 209 - protein, 27 aa, Coronavirinae
EVFAQVKQMYKTPTLKYFGGFNFSQIL

SEQ ID NO: 210 - protein, 27 aa, Coronavirinae
EVFAQVKQMYKTPAIKDFGGFNFSQIL

SEQ ID NO: 211 - protein, 18 aa, Coronavirinae
SFIEDLLFNKVTLADAGF

SEQ ID NO: 212 - protein, 18 aa, Coronavirinae
SAIEDLLFNKVRLSDVGF

SEQ ID NO: 213 - protein, 18 aa, Coronavirinae
SLLEDLLFNKVKLSDVGF

SEQ ID NO: 214 - protein, 18 aa, Coronavirinae
SAIEDLLFSKVKLADVGF

SEQ ID NO: 215 - protein, 18 aa, Coronavirinae
SAIEDLLFDKVKLSDVGF

SEQ ID NO: 216 - protein, 17 aa, Filoviridae
GAAIGLAWIPYFGPAAE

SEQ ID NO: 217 - protein, 17 aa, Filoviridae
GAAVGLAWIPYFGPAAE

SEQ ID NO: 218 - protein, 17 aa, Filoviridae
GAAAGLAWIPYFGPAAE

SEQ ID NO: 219 - protein, 17 aa, Filoviridae
DLAAGLSWIPFFGPGIE

SEQ ID NO: 220 - protein, 17 aa, Filoviridae
HNAAGIAWIPYFGPGAE

SEQ ID NO: 221 - protein, 23 aa, Hiv1
AVGLGALFLGFLGAAGSTMGAAS

SEQ ID NO: 222 - protein, 12 aa, Hiv1
LTLTGQARQLLS

SEQ ID NO: 223 - protein, 17 aa, Hiv1
GIVQQQSNLLQAIEAQQ

SEQ ID NO: 224 - protein, 33 aa, Hiv1
GLGAMFLGFLGAAGSTMGAASLTLTVQARQLLS

SEQ ID NO: 225 - protein, 33 aa, Hiv1
GIGAMFLGLLSAAGSTMSAAAITLTVQTRQLLS

SEQ ID NO: 226 - protein, 33 aa, Hiv1
GIGAMFLGLLSAAGSTMGAAAITLTVQTRQLLS

SEQ ID NO: 227 - protein, 33 aa, Hiv1
GIGAVFLGFLGAAGSTMGAASITLTVQARQLLS

SEQ ID NO: 228 - protein, 33 aa, Hiv1
GVGALFLGFLSAAGSTMGAASITLTVQARQLLS

SEQ ID NO: 229 - protein, 33 aa, Hiv1
GIGAMILGFLGAAGSTMGAASITLTVQARQLLS

SEQ ID NO: 230 - protein, 33 aa, Hiv1
GLGAMFLGFLGAAGSTMGAASITLTVQARQLLS

SEQ ID NO: 231 - protein, 33 aa, Hiv1
GFGAMFLGFLGAAGSTMGAASITLTVQARQLLS

SEQ ID NO: 232 - protein, 33 aa, Hiv1
TLGAMFLGFLGAAGSTMGAASMTLTVQARQLLS

SEQ ID NO: 233 - protein, 33 aa, Hiv1
GLGAVFLGFLGAAGSTMGAASITLTVQARQLLS

SEQ ID NO: 234 - protein, 33 aa, Hiv1
TIGAMFLGFLGAAGSTMGAASITLTVQARRLLS

SEQ ID NO: 235 - protein, 33 aa, Hiv1
TIGAMFLGFLGAAGSTMGAASMTLTVQARLLLS

SEQ ID NO: 236 - protein, 33 aa, Hiv1
TLGAMFLGFLGAAGSTMGAASMTLTVQARLLFS

SEQ ID NO: 237 - protein, 33 aa, Hiv1
TLGAMFLGFLGAAGSTMGAASLTLTVQARLLLS

SEQ ID NO: 238 - protein, 33 aa, Hiv1
GVGAMFLGFLGAAGSTMGAASLTLTVQARQLLS

SEQ ID NO: 239 - protein, 33 aa, Hiv1
GLGAMFLGFLGAAGSTMGAASITLTVQARLLLS

SEQ ID NO: 240 - protein, 33 aa, Hiv1
TLGAVFLGFLGAAGSTMGAASLTLTVQARLLLS

SEQ ID NO: 241 - protein, 33 aa, Hiv1
GIGAVFLGFLGAAGSTMGAASITLTVQARKLLS

SEQ ID NO: 242 - protein, 33 aa, Hiv1
GIGALFLGFLGAAGSTMGAASVTLTVQARQLLS

SEQ ID NO: 243 - protein, 33 aa, Hiv1
GLGALFLGFLGAAGSTMGAASVTLTVQARQLLS

SEQ ID NO: 244 - protein, 33 aa, Hiv1
GIGAMFLGFLGAAGSTMGAASITLTVQARLLLS

SEQ ID NO: 245 - protein, 33 aa, Hiv1
GIGAMFLGFLGAAGSTMGAASVTLTVQARLLLS

SEQ ID NO: 246 - protein, 33 aa, Hiv1
AIGALFLGFLGAAGSTMGAASVTLTVQARLLLS

SEQ ID NO: 247 - protein, 33 aa, Hiv1
TLGAMFLGFLGAAGSTMGAASLTLTVQARQLLS

SEQ ID NO: 248 - protein, 33 aa, Hiv1
GIGALFLGFLGAAGSTMGAASMTLTVQARQLLS

SEQ ID NO: 249 - protein, 33 aa, Hiv1
GIGAMFLGFLGAAGSTMGAASLTLTVQARQLLS

SEQ ID NO: 250 - protein, 33 aa, Hiv1
GIGAVFLGFLGAAGSTMGAASMTLTVQARLLLS

SEQ ID NO: 251 - protein, 33 aa, Hiv1
GIGALFLGFLGAAGSTMGAASLTLTVQARQLLS

SEQ ID NO: 252 - protein, 33 aa, Hiv1
GIGAVFLGILGAAGSTMGAASITLTVQARQLLS

SEQ ID NO: 253 - protein, 33 aa, Hiv1
GIGAVFLGFLGAAGSTMGAASVTLTVQARQLLF

SEQ ID NO: 254 - protein, 33 aa, Hiv1
GLGAMFFGFLGAAGSTMGAASVTLTVQARQLLS

SEQ ID NO: 255 - protein, 33 aa, Hiv1
GIGALFLGFLGAAGSTMGAASITLTVQARLLLS

SEQ ID NO: 256 - protein, 33 aa, Hiv1
GLGALFVGFLGAAGSTMGAASITLTVQARQLLS

SEQ ID NO: 257 - protein, 33 aa, Hiv1
GIGALFLGFLGTAGSTMGAASVTLTVQARQLLS

SEQ ID NO: 258 - protein, 33 aa, Hiv1
GIGAMIFGFLGAAGSTMGAASITLTVQARQLLS

SEQ ID NO: 259 - protein, 33 aa, Hiv1
GLGAVLLGFLGTAGSTMGAASLTLTVQVRQLLS

SEQ ID NO: 260 - protein, 33 aa, Hiv1
GIGAVLFGFLGAAGSTMGAASITLTVQVRQLLS

SEQ ID NO: 261 - protein, 33 aa, Hiv1
GLGALFLGFLGAAGSTMGAASLTLTGQARQLLS

SEQ ID NO: 262 - protein, 33 aa, Hiv1
GTLGAMFLGFLGAAGSTMGAASMTLTVQARQLL

SEQ ID NO: 263 - protein, 33 aa, Hiv1
GTIGAMFLGFLGAAGSTMGAASITLTVQARRLL

SEQ ID NO: 264 - protein, 33 aa, Hiv1
GTIGAMFLGFLGAAGSTMGAASMTLTVQARLLL

SEQ ID NO: 265 - protein, 33 aa, Hiv1
IGALFLGFLGAAGSTMGAASVTLTVQARLLLSG

SEQ ID NO: 266 - protein, 23 aa, Lentivirus
AVGMVIFLLVLAIMAMTASVTAA

SEQ ID NO: 267 - protein, 20 aa, Lentivirus
FGISAIVAAIVAATAIAASA

SEQ ID NO: 268 - protein, 21 aa, Lentivirus
TLALVTATTAGLIGTTTGTSA

SEQ ID NO: 269 - protein, 21 aa, Lentivirus
HVMLALATVLSMAGAGTGATA

SEQ ID NO: 270 - protein, 20 aa, Lentivirus
GIGLVIMLVTMAIVAAAGAS

SEQ ID NO: 271 - protein, 20 aa, Lentivirus
GVMVLGFLGFLAMAGSAMGA

SEQ ID NO: 272 - protein, 20 aa, Lentivirus
GVFVLGFLGFLATAGSAMGA

SEQ ID NO: 273 - protein, 19 aa, Lentivirus
GAIVLGLLGFLGLAGSAMG

SEQ ID NO: 274 - protein, 28 aa, Lentivirus
GIGLVIVLAIMAIIAAAGAGLGVANAVQ

SEQ ID NO: 275 - protein, 25 aa, influenza virus with synthetic peptide tail GGEKEKEK
GLFGAIAGFIENGWEGCGGEKEKEK

SEQ ID NO: 276 - protein, 18 aa, Ebolavirus
LLNRKAIDFLLQRWGGTC

SEQ ID NO: 277 - protein, 18 aa, Ebolavirus
ILNRKAIDFLLQRWGGTC

SEQ ID NO: 278 - protein, 18 aa, Ebolavirus
ILNRKAIDFLLQRRGGTC

SEQ ID NO: 279 - protein, 29 aa, SARs coronavirus
AEVQIDRLITGRLQSLQTYVCGGEKEKEK

SEQ ID NO: 280 - protein, 26 aa, Filovirus
GAAIGLAWIPYFGPAAECGGEKEKEK

SEQ ID NO: 281 - protein, 25 aa, influenza virus with synthetic peptide tail GGEKEKEK
GLFGAIAGFIENGWEGGGGEKEKEK